(19) United States
(12) Patent Application Publication
(10) Pub. No.: US 2017/0074855 A1
(43) Pub. Date: Mar. 16, 2017

(54) Scaffold Data Storage and Target Detection in a Sample Using a Nanopore

(71) Applicant: Two Pore Guys, Inc., Santa Cruz, CA (US)

(72) Inventors: Trevor J. Morin, Santa Cruz, CA (US); Daniel A. Heller, Santa Cruz, CA (US); William B. Dunbar, Santa Cruz, CA (US)

(21) Appl. No.: 15/310,426

(22) PCT Filed: May 15, 2015
(86) PCT No.: PCT/US2015/031242

§ 371 Date: Nov. 10, 2016

Related U.S. Application Data
(60) Provisional application No. 61/993,985, filed on May 15, 2014.

(51) Int. Cl. G01N 33/487; G01N 33/543

(52) U.S. Cl. CPC G01N 33/48721; G01N 33/54306

(57) ABSTRACT

Provided are methods and compositions for detecting a target analyte suspected to be present in a sample with background molecules using a nanopore device. A plurality of probes for polymer scaffold identification or for target analyte binding and detection are provided. Also provided are methods and compositions for storing data on a polymer scaffold, and accessing the data using a nanopore device and binding probes.

### CROSS-REFERENCE TO RELATED APPLICATIONS

This application claims the benefit under 35 U.S.C §119(e) to U.S. provisional application No. 61/993,985, filed May 15, 2014, the contents of which are incorporated by reference in their entirety.

### BACKGROUND

Methods and systems for highly sensitive detection of analytes, such as molecules, tumor cells, pathogenic organisms, have broad applications, in particular, clinically, for pathogen detection and disease diagnosis, for instance. Additionally, such detection can: allow for the personalization of medical treatments and health programs; facilitate the search for effective pharmaceutical drug compounds and biotherapeutics; and enable clinicians to identify abnormal hormones, ions, proteins, or other molecules produced by a patient's body and/or identify the presence of poisons, illegal drugs, or other harmful chemicals ingested or injected into a patient.

Nanopores have shown great promise as a low cost, low-energy, tiny sensor capable of detecting biological molecules for a range of purposes, from sequencing DNA to detecting target analytes that indicate the presence of diseases, pathogens, or other biomarkers of interest. A nanopore device can detect a molecule passing through a nanopore by a current impedance signal. The problem has been that the current impedance (or equivalent, e.g., current or voltage) information produced by a nanopore does not have sufficient resolution to distinguish the molecule. Several different molecules that pass through produce such similar electrical signals, so that it is nearly impossible to discriminate one from another.

There have been attempts to bind additional molecules to target analytes, so as to create larger current impedance (or equivalent) signals that can then be more easily identified, but this has shown to be insufficient when combined with original (even filtered) natural fluids (blood, saliva, urine, etc.), which have a vast population of background molecules that produce false positives, generating a high error rate of detection. Adding sophisticated sample preparation to filter out non-target markers technically helps, but the added cost and complexity exceeds that of existing non-nanopore technologies in use today. What is needed, therefore are methods and compositions for improved accuracy of detection of biological molecules and other analytes from a sample.

Research on semiconductors have dramatically improved the capacity of data storage in silicon devices. However, given the even faster growth of need of data storage, the semiconductor industry is facing new challenges. Nucleotides, such as DNA and RNA, store genetic information in large scales. Therefore, polynucleotides or other polymers can be potentially used for data storage. However, decoding the information in a polymer presents greater challenges than reading a conventional computer memory which is typically read by optical or electromagnetic means.

Nanopores have been looked into for reading information encoded in a single molecule. When a molecule passes through the nanopore, its structure can potentially be characterized by changes of the ionic currents (or equivalent signals) caused by the passing. What is needed, therefore, are improved methods and compositions storing and decoding information on a polynucleotide or other polymer using a nanopore device.

### SUMMARY

Various aspects disclosed herein can fulfill one or more of the above-mentioned needs. The systems and methods described herein each have several aspects, no single one of which is solely responsible for its desirable attributes. Without limiting the scope of this disclosure as expressed by the claims that follow, the more prominent features will now be discussed briefly. After considering this discussion, and particularly after reading the section entitled “Detailed Description,” one will understand how the sample features described herein provide for improved systems and methods.

In an embodiment, the present disclosure provides a method for detecting a target analyte suspected to be present in a mixed sample, the method comprising: (a) loading a polymer scaffold, a fusion molecule or compound, a label, and a mixed sample suspected to contain a target analyte into a device comprising a nanopore that separates an interior space of the device into two volumes, under conditions that allow said label to bind to said polymer scaffold, that allow said fusion molecule or compound to bind to said polymer scaffold, and that allow said fusion molecule or compound to bind to said target analyte, wherein said polymer scaffold comprises at least one fusion molecule binding domain capable of binding to the fusion molecule or compound, wherein said polymer scaffold comprises at least one label or label binding domain capable of binding to the label, wherein said fusion molecule or compound comprises a target binding domain capable of binding to the target analyte, and wherein said fusion molecule comprises a scaffold binding domain capable of binding to the polymer scaffold at a first target, and wherein said label comprises a scaffold binding domain capable of binding to the polymer scaffold at a second target; (b) configuring the device to pass the polymer scaffold in any orientation through the nanopore from one volume to the other volume; and (c) collecting a electrical signal correlated to passage of said polymeric scaffold in any orientation through the nanopore.

In certain embodiments, the polymer scaffold is dsDNA. In some embodiments, the polymer scaffold has a plurality of ordered label binding domains for increased resolution of detection of the polymer scaffold in a bulk sample with a plurality of background molecules. In some embodiments, the polymer scaffold has a plurality of unique fusion molecule binding domains to allow multiplexing of target detection.

In some embodiments, the fusion molecule provides a provides a unique and detectable electrical signal in a bound state as compared to an unbound state upon translocation through the nanopore when bound to said polymer scaffold. In some embodiments, the fusion molecule comprises PNA bound to a molecule comprising a target binding moiety.

In some embodiments, the label comprises PNA. In certain embodiments, the PNA is bound to a detectable tag, such as a PEG. In certain embodiments, the size, shape, and or charge of the detectable tag can be modified to increase resolution based on current impedance (or equivalent signals) in a pore of a specific shape or size.

Also provided are methods of analyzing data from a nanopore device to detect the presence of a target analyte in a mixed sample, the method comprising: (a) obtaining an electrical signal from an event generated by a nanopore analysis of a mixture, wherein said mixture comprises a sample suspected of containing a target analyte, a polymer scaffold comprising at least one fusion molecule binding domain and at least one label binding domain or label, and a fusion molecule capable of binding said fusion molecule binding domain and said target analyte; (b) analyzing said electrical signal to detect the presence of a first signature curve indicating detection of a label attached to the polymer scaffold; and (c) analyzing said electrical signal to detect the presence of a second signature curve indicating detection of a target analyte attached to said polymer scaffold.

Also provided are compositions for enhancing detection of analytes form a mixed sample using a nanopore. Thus, in an embodiment, provided is a polymeric scaffold comprising at least one fusion molecule binding domain and at least one label binding domain or label. In certain embodiments, the polymeric scaffold comprises a plurality of fusion molecule binding domains for multiplex analysis of analytes. In other embodiments, the polymeric scaffold comprises a plurality of fusion molecule binding domains for increased resolution of detection of a single analyte. In an embodiment, the polymeric scaffold comprises a plurality of label binding domains for increased resolution of identification of the polymeric scaffold.

In some embodiments, provided is a polymeric scaffold bound to a plurality of probes. In an embodiment, the probe is a fusion molecule. In another embodiment, the probe is a label. In some embodiments, the fusion molecule has a target analyte binding moiety. In some embodiments, the fusion molecule is attached to the polymeric scaffold and to a target analyte. In some embodiments, the fusion molecule is attached to the target analyte through an intermediary.

Also provided are methods of encoding one or more bit(s) of information by placing one or more molecules along a polymer in such a fashion that the original information can be retrieved by passing the polymer through a nanopore and examining the current impedance signatures curves.

Also provided are kits, packages or mixtures that detect the presence of a target molecule or particle. In an embodiment, the kit comprises a polymer scaffold comprising at least one fusion molecule binding domain and at least one label binding domain, a label capable of binding to said binding domain, and a fusion molecule capable of binding to a target ligand and to said fusion molecule binding domain. In some aspects, the kit, package or mixture further comprises a sample suspected of containing the target molecule or particle. In some aspects, the sample further comprises a detectable label capable of binding to the target molecule, particle, ligand/target complex, or ligand/particle complex.

Also provided are method of analyzing data to detect the presence of a target analyte in a mixed sample, comprising (a) obtaining an electrical signal from an event generated by a nanopore analysis of a mixture, wherein said mixture comprises a sample suspected of containing a target analyte, a polymer scaffold comprising at least one fusion molecule binding domain and at least one label binding domain or label, and a fusion molecule capable of binding said fusion molecule binding domain and said target analyte; (b) analyzing said electrical signal to detect the presence of a first signature curve indicating detection of a label attached to the polymer scaffold; and (c) analyzing said electrical signal to detect the presence of a second signature curve indicating detection of a target analyte attached to said polymer scaffold.

In addition, provided is a method for identifying binding sequences on a polymer scaffold, comprising: (a) providing a polymer scaffold comprising a label binding domain; (b) loading said polymer scaffold and a label configured to bind to said label binding domain into a device comprising a nanopore that separates an interior space of the device into two volumes, under conditions that allow said label to bind to said label binding sequence; (c) configuring the device to pass the polymer scaffold through the nanopore from one volume to the other volume; and (d) collecting an electrical signal correlated to passage of said polymeric scaffold through the nanopore.

Also provided are kits, packages or mixtures to store and/or read information on a polymer scaffold. In an embodiment, the kit comprises two or more labels each having different size, charge and/or shape and a polymer scaffold encoding information to be read. In some embodiments, the kit further comprises a nanopore device comprising a nanopore that separates and connects two volumes in the nanopore device, wherein the nanopore device is configured to identify each of the labels when the label is bound to said polymeric scaffold and said polymeric scaffold translocates through said nanopore.

### BRIEF DESCRIPTION OF THE DRAWINGS

Provided as embodiments of this disclosure are drawings that illustrate features by exemplification only, and not limitation.

FIG. 1 illustrates how a nanopore is configured to detect ligands bound to a nucleotide.

FIGS. 2A and 2B shows a PNA ligand that has been modified as to increase ligand size, and therefore facilitate detection. FIG. 2C shows a DNA scaffold that contains a reactive moiety and conjugates to a molecule that has compatible reactivity for covalent coupling

FIG. 3A illustrates the detection of a target molecule or particle with fusion molecules according to an embodiment of the method. FIG. 3B

FIG. 4 shows representative and idealized current profiles of three example molecules, demonstrating that binding between a target molecule (or particle) and a fusion molecule can be detected when passing through a nanopore, since it has a different current profile, compared to that of the fusion molecule alone or the DNA alone. Specifically, FIG. 4A shows current profiles consistent with higher salt concentrations (>0.4 M KCI, for example at 1M KCI) in the experimental buffer and a positive applied voltage, generating a positive current flow through the pore. By another example, FIG. 4B shows current profiles consistent with lower salt concentrations (<0.4 M KCI, for example at 100 mM KCI) in the experimental buffer and again at a positive applied voltage. By another example, FIG. 4C shows current profiles consistent with lower salt concentrations (<0.4 M KCI, for example at 100 mM KCI) in the experimental buffer and a negative applied voltage.

FIG. 5 illustrates the multiplexing capability of the present technology by including different binding motifs in the polymer scaffold. Such multiplexing can be accomplished with one nanopore or more than one nanopore.

FIG. 6 provides the illustration of a more specific example, where a double-stranded DNA is used as the polymer scaffold, and a human immunodeficiency virus (HIV) envelope protein is used as the ligand. The combination is used to detect an anti-HIV antibody.

FIG. 7 illustrates a nanopore device with at least two pores separating multiple chambers. Specifically, FIG. 7A is a schematic of a dual-pore chip and a dual-amplifier electronics configuration for independent voltage control (V₁ or V₂) and current measurement (l₁, or l₂) of each pore. Three chambers, A-C, are shown and are volumetrically separated except by common pores. FIG. 7B is a schematic where electrically, V₁ and V₂ are principally applied across the resistance of each nanopore by constructing a device that minimizes all access resistances to effectively decouple l₂ and l₂. FIG. 7C depicts a schematic in which competing voltages are used for control, with arrows showing the direction of each voltage force.

FIG. 8 illustrates a nanopore device having one pore connecting two chambers and example results from its use. Specifically, panel (a) depicts a schematic diagram of the nanopore device. Panel (b) depicts a representative current trace showing a blockade event resulting from the passage of a double-stranded DNA passing through the pore. The current amplitude shift amount (Δ/=l₀−l_{B}) and duration to are used to quantify the passage event. Panel (c) depicts a scatter plot showing the change in current amount (Δl) vs. translocation time (t_{D}) for all blockade events recorded over 16 minutes.

FIG. 9 depict current traces measured within an embodiment of a nanopore device fabricated in accordance with the present invention. The provided current traces show that unbound dsDNA causes current enhancement events at KCI concentrations below 0.4 M. Current enhancements appeared as downward shifts in the provided experiment, since the voltage and current are both negative (as in FIG. 3C). Specifically, in DNA alone control experiments using a 10-11 nm diameter pore in 0.1M KCI at −200 mV, 5.6 kb dsDNA scaffold (panel (a)) causes brief current enhancement events that are 50-70 pA in amplitude and 10-200 microseconds in duration. Likewise, 48 kb Lambda DNA (panel (b)) causes current enhancement events 50-70 pA in amplitude and 50-2000 microseconds in duration.

FIG. 10 illustrates a gel showing the sequence specificity of binding of a bisPNA to a dsDNA polymer scaffold.

FIG. 11 shows representative electrical signals from nanopore detection of a polymer scaffold (panel(a)) not bound to bisPNA, and panels (b), (c) bound to bisPNA.

FIG. 12 is a gel showing binding of PNA without (lane 2) or with a detectable tag of PEG 5 k (lane 3) or PEG 10 k (lane 4).

FIG. 13 shows representative electrical signals from nanopore detection of a polymer scaffold bound to (panel (a)) PNA alone, (panel (b)) PNA with a PEG 5 k detectable tag, or (panel (c)) PNA with a PEG 10 k detectable tag.

FIG. 14 shows the results of a gel shift assay shows that a single (lane 3) or two (lane 4) gammaPNA-PEG 5 kDa can bind to the same fragment molecule.

FIG. 15 shows the results of a gel shift assay shows that one (lane 3) or two (lane 4) monostrepatavidin proteins can bind to a single dsDNA polymer scaffold with multiple label (monostreptavidin) binding sites.

FIG. 16 illustrates detection of multiple labels on a dsDNA scaffold. Panel (a) shows a gel shift assay. Panel (a) is an image from a DNA-(PNA-biotin)-Neutravidin (DPN) EMSA in labeling buffer, with the following lanes (left to right): sizing ladder with top rung 5 kb; 5.6 kb DNA only; DNA-PNA with 3×, 7×, 16× and 36× excess Neutravidin to biotin; and DNA-PNA. Panel (b) is a schematic of one PNA-biotin-Neutravidin region on the 5.6 kb dsDNA scaffold, and a representative translocation event recorded from each of three consecutive experiments using the same pore at 200 mV in 1M KCI: DNA alone, Neutravidin alone, and then DPN complexes with 10× excess Neutravidin to biotin. Panel (c) is a scatter plot of ΔG versus duration for the three consecutive experiments (D, N, and DPN). Panel (d) is a horizontal probability histogram of ΔG for the three data sets, with the inset histogram for the 578 DPN events with duration longer than 0.08 ms.

FIG. 17 shows a prototype illustration of an electrical signal generated upon the translocation of a polymer scaffold with PNA molecules attached to 5K PEGs on either end of the polymer scaffold, with a fusion molecules and target analyte in the middle, through the nanopore.

FIG. 18 shows a dsDNA scaffold with events 0.1-0.5 ms, and with a single antibody acting as a label at one end, and the absence or presence of a separate target analyte antibody at the other end. Event signatures have a single “spike” when only the label antibody is present, and two “spikes” when the target analyte antibody is present, signaling detection of the target for that molecule.

FIG. 19 shows a dsDNA scaffold with events 0.5-10 ms, and with a single antibody acting as a label at one end, and the absence or presence of a separate target analyte antibody at the other end. Event signatures have a single “spike” when only the label antibody is present, and two “spikes” when the target analyte antibody is present, signaling detection of the target for that molecule.

Some or all of the figures are schematic representations for exemplification; hence, they do not necessarily depict the actual relative sizes or locations of the elements shown. The figures are presented for the purpose of illustrating one or more embodiments with the explicit understanding that they will not be used to limit the scope or the meaning of the claims that follow below.

### DETAILED DESCRIPTION

Throughout this application, the text refers to various embodiments of the present devices, compositions, systems, and methods. The various embodiments described are meant to provide a variety of illustrative examples and should not be construed as descriptions of alternative species. Rather, it should be noted that the descriptions of various embodiments provided may be of overlapping scope. The embodiments discussed herein are merely illustrative and are not meant to limit the scope of the present invention.

Also throughout this disclosure, various publications, patents and published patent specifications are referenced by an identifying citation. The disclosures of these publications, patents and published patent specifications are hereby incorporated by reference into the present disclosure in their entireties.

As used in the specification and claims, the singular form “a”, “an” and “the” include plural references unless the context clearly dictates otherwise. For example, the term “an electrode” includes a plurality of electrodes, including mixtures thereof

As used herein, the term “comprising” is intended to mean that the systems, devices, and methods include the recited components or steps, but not excluding others. “Consisting essentially of” when used to define systems, devices, and methods, shall mean excluding other components or steps of any essential significance to the combination. “Consisting of” shall mean excluding other components or steps. Embodiments defined by each of these transition terms are within the scope of this invention.

All numerical designations, e.g., distance, size, temperature, time, voltage and concentration, including ranges, are approximations which are varied (+) or (−) by increments of 0.1. It is to be understood, although not always explicitly stated that all numerical designations are preceded by the term “about”. It also is to be understood, although not always explicitly stated, that the components described herein are merely exemplary, and that equivalents of such are known in the art.

As used herein, “a device comprising a nanopore that separates an interior space” shall refer to a device having a pore that comprises an opening within a structure, the structure separating an interior space into more than one volume or chamber.

As used herein, the term “scaffold” or “polymer scaffold” refers to a charged polymer capable of binding probes and translocating through a pore upon application of voltage. In some aspects, the polymer scaffold comprises a deoxyribonucleic acid (DNA), a ribonucleic acid (RNA), a peptide nucleic acid (PNA), a DNA/RNA hybrid, or a polypeptide. The scaffold can also be a chemically synthesized polymer, and not a naturally occurring or biological molecule. In a preferred embodiment, the polymer scaffold is dsDNA to allow more predictable signals upon translocation through the nanopore and reduce secondary structure present in ssDNA or RNA. The polymer scaffold comprises probe binding domains, e.g., label binding domains and/or fusion molecule binding domains. These domains can reside on the ends of the DNA as chemical modification to which labels or analyte detection molecules are chemically tethered or bound.

As used herein, the term “binding domain” when referring to a segment on the polymer scaffold, e.g., a fusion molecule binding domain or a label binding domain, refers to a domain that binds under stringent conditions to another molecule or compound. In the case of several embodiments of this invention, the binding domain comprises a specific sequence on the polymer scaffold which binds to a probe. Other embodiments, the binding domain is a modification to the end of the scaffold to enable probe attachment or binding. The address/bit location of each binding domain can be determined by detection of the binding of the probes to the polymer scaffold in a nanopore device.

As used herein, the term “probes” refers to molecules or compounds that bind to a binding domain on or at the terminal ends of a polymer scaffold. In several embodiments of this invention, the probes are fusion molecules or compounds, or labels.

As used herein, the term “labels” refer to molecules or compounds that bind to a specific label binding domain on or at the terminal ends of a polymer scaffold. These compounds are configured to be detectable by a nanopore by measuring current impedance. Labels can comprise “detectable tags” which are detectable in a nanopore when bound to a polymer scaffold due to their size, shape, or charge providing a detectable effect on current impedance. Thus, the detectable tag can be used to enhance resolution of detection of the label in a nanopore or to provide unique characteristics for identification in a nanopore via a unique electrical signal. The detectable tag can be attached, either covalently or non-covalently, to the label or directly to the polymer scaffold.

As used herein, the term “fusion molecule” refers to molecules or compounds that bind to a specific fusion molecule binding domain on, or bind or react with chemical groups at the termini of a polymer scaffold, and also bind to a target analyte. Upon translocation through the nanopore, a fusion molecule bound to the polymer scaffold can generate an electrical signal that is capable of discriminating whether or not the fusion molecule is bound to a target analyte. In this way, a target analyte in a solution can be detected and/or quantified.

As used herein, the term “target analyte” refers to a molecule, compound, virus, cell, or other entity of interest to be detected in a sample. The target analyte may be detected by binding to an analyte binding domain on a fusion molecule attached to a polymer scaffold that translocates through a nanopore, providing a defined electrical signal.

As used herein, the term “electrical signature” encompasses a series of data collected on current, impedance/resistance, or voltage over time depending on configuration of the electronic circuitry. Conventionally, current is measured in a “voltage clamp” configuration; voltage is measured in a “current clamp” configuration, and resistance measurements can be derived in either configuration using Ohm's law V=IR. Impedance can also be generated by measured from current or voltage data collected from the nanopore device. Types of electrical signals referenced herein include current signatures and current impedance signatures, although various other electrical signatures may be used to detect particles in a nanopore.

As used herein, the term “nanopore” refers to an opening (hole or channel) of sufficient size to allow the passage of particularly sized polymers. Voltage is applied to drive negatively charged polymers through the nanopore.

As used herein, the term “sensor” refers to a device that collects a signal from a nanopore device. In many embodiments, the sensor includes a pair of electrodes placed at two sides of a pore to measure an ionic current across the pore when a molecule or other entity, in particular a polymer scaffold, moves through the pore. In addition to the electrodes, an additional sensor, e.g., an optical sensor, may be to detect an optical signal in the nanopore device. Other sensors may be used to detect such properties as current blockade, electron tunneling current, charge-induced field effect, nanopore transit time, optical signal, light scattering, and plasmon resonance.

As used herein, the term “current measurement” refers to a series of measurements of current flow at an applied voltage through the nanopore over time. The current is expressed as an x,y value where x represents a point in time, and y represents the amount of current impeded in the channel. Current measurement is an electrical signal related to current impedance/resistance and voltage (other electrical signals) through Ohm's law.

As used herein, the term “open channel” refers to the baseline level of current through a nanopore channel within a noise range where the current does not deviate from a threshold of value defined by the analysis software.

As used herein, the term “event” refers to a set of current impedance measurements that begins when the y value of a current measurement deviates from the open channel value by a defined threshold, and ends when the y value returns to within a threshold of the open channel value.

As used herein, the term “current impedance signature” refers to a collection of current measurements where the first such measurement begins when the value of y exceeds a given threshold defined by the software, and ends when the value of y returns past that same threshold. This threshold may be used to identify multiple signatures within an event (i.e., since a polymer may have one or more molecules attached to it, an event may contain one or more signatures).

As used herein, the term “signature curve” refers to the product of a mathematical formula applied to all the x,y points in a single signature. This formula may be as simple as a simple average of all the points (yielding a single line at y), or as a moving average of every N number of points (yielding a simple curve), or another mathematical formula. Step fitting algorithms are another example of a formula to apply to each signature. The number of steps or their properties can be used to infer properties about the signature curve or curves. (See, e.g., C Raillon, P Granjon, M Graf, L J Steinbock, and A Radenovic. Fast and automatic processing of multi-level events in nanopore translocation experiments. Nanoscale, 4(16):4916, 2012, incorporated by reference in entirety). Since nanopores are inherently non-deterministic, electrical signals may vary considerably each time the same type of molecule passes through. Therefore, the software that analyzes measurements may employ enough flexibility to assure a consistent signature curve each time the same molecule is read.

As used herein, the term “optical sensor” refers to an apparatus that captures light within a fixed field of view that may reside at or adjacent to the nanopore.

As used herein, the term “optical event” refers to a set of optical measurements captured by the sensor from a single polymer that may contain one or more tagged molecules. Because the sensor cannot discern between the beginning and end of a polymer using optics, the ends of the polymer may be detected by using current impedance measurements to determine when a polymer enters (when the measurement's y value exceeds the open channel threshold, or by adding tagged molecules that will produce a known optical measurement to the each end of the polymer.

As used herein, the term “optical measurement” refers to a value obtained by that optical sensor within a fixed period of time. This measurement may include, but not be limited to, one or more of individual values, such as color, luminescence, and intensity.

As used herein, the term “symbol” refers to the assembly of one or more optical signatures within an event so as to comprise a single abstraction. E.g., “red, green, red, green” may equate to the letter “A.”

### Polymer Scaffold Identification and Decoding

The present disclosure provides methods and systems for molecular detection and quantitation. In addition, the methods and systems can also be configured to measure the affinity of a molecule binding with another molecule. Further, such detection, quantitation, and measurement can be carried out in a multiplexed manner, greatly increasing its efficiency.

The present disclosure, in an embodiment, provides devices and methods for identifying a polymer scaffold, such as a DNA, RNA, PNA or polypeptide molecule, using a nanopore. The methods employ a plurality of detectable labels that specifically bind to a particular sequence (referred to as a “label binding domain”) on the polymer scaffold. The labels can differ from each other by size, shape, or charge. Therefore, when a polymer scaffold bound to a set of labels is passed through a suitably configured nanopore, the labels can be identified or at least distinguished from each other by measuring the current impedance as each label passes through the nanopore. Orientation of the polymer scaffold as it translocates through the nanopore is not limited to a specific direction, as electrical signals for labels may be identified based on translocation in either orientation.

By virtue of the binding specificity between the detectable labels and the label binding domains, the relative locations and order of the label binding domains on the polymer scaffold can be derived from the bound labels that generate a unique current impedance in the nanopore. Thus, the nanopore device does not need to identify each monomer of the entire polymer scaffold or even a portion of the polymer scaffold. Therefore, if a polymer scaffold is encoded with information in a format of sequences of label binding domains, the detection of the labels bound to the label binding domains “decodes” such information.

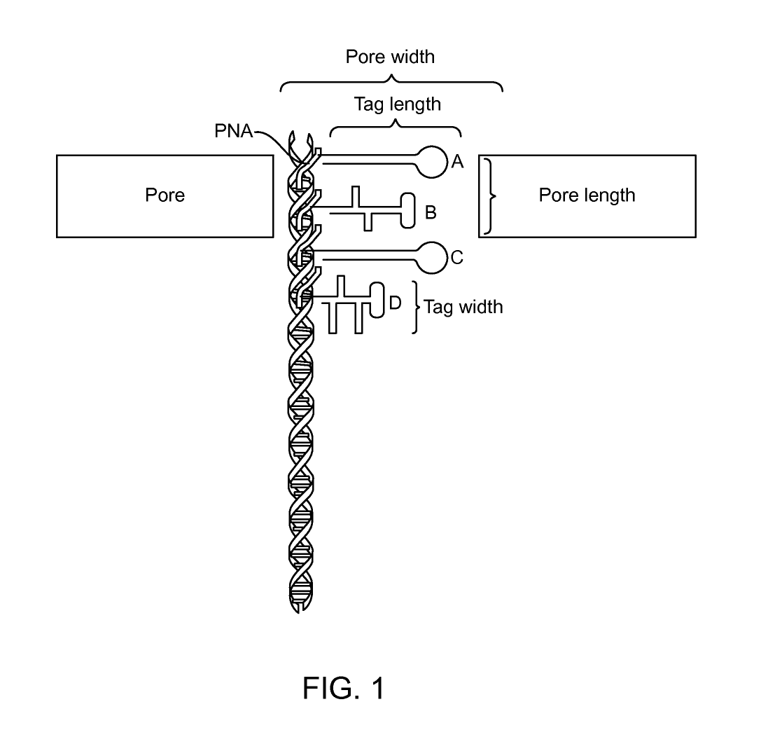

As illustrated in FIG. 1, in certain embodiments, labels A, B, C and D each specifically binds to a label binding domain on a DNA molecule. In the embodiment shown, each label comprises a PNA molecule attached to a detectable tag. These labels can be identified and distinguished from each other by their current impedance when passing through the nanopore. This current impedance is affected by width, length, size and/or charge of the label. In the embodiment provided in FIG. 1, these parameters are determined by the width, length, size, and/or charge of the detectable tag attached to the PNA molecule. Thus, each label may provide a unique electrical signal upon passage through the nanopore, allowing identification of each label bound to the polymer scaffold and therefore to each label binding domain present on the polymer scaffold. The PNA molecule comprises a sequence complementary to the label binding domain on the double-stranded DNA. Identification of the labels shown in FIG. 1 leads to identification of the sequence of label binding domains, A-B-C-D. If the polymer scaffold entered into the opposite orientation, the sequence would still be detected as D-C-B-A, and provide information regarding the location and identity of the label binding domains on the polymer scaffold. In practice, the labels will likely be spaced apart more than they appear in the figure. On the order of 10 s to 100 s to 1000 s of basepairs apart.

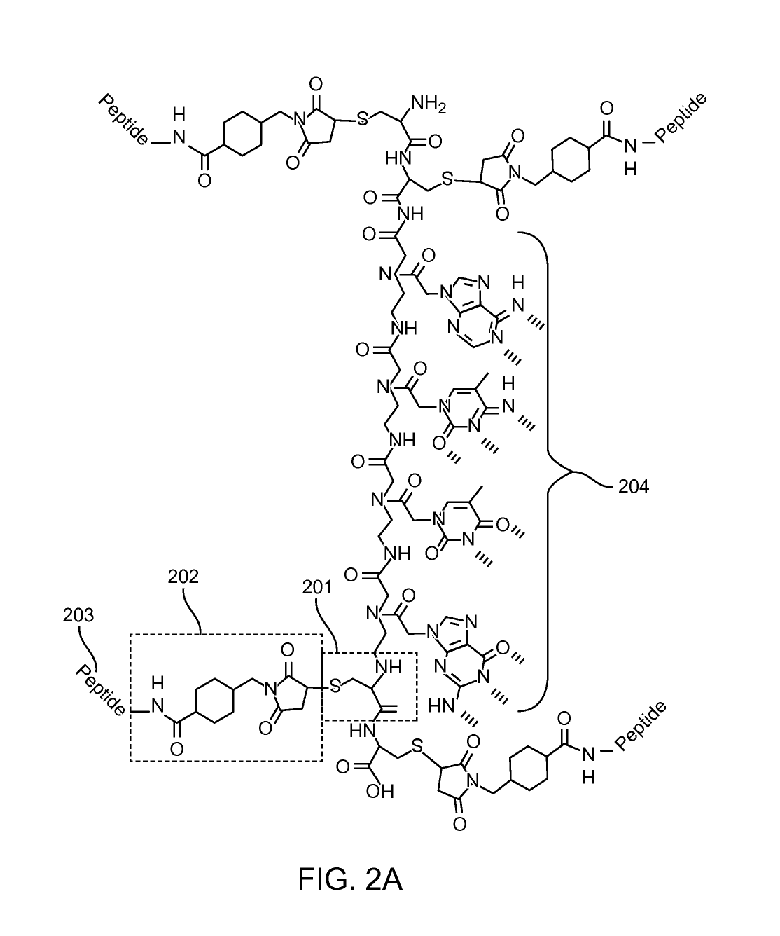

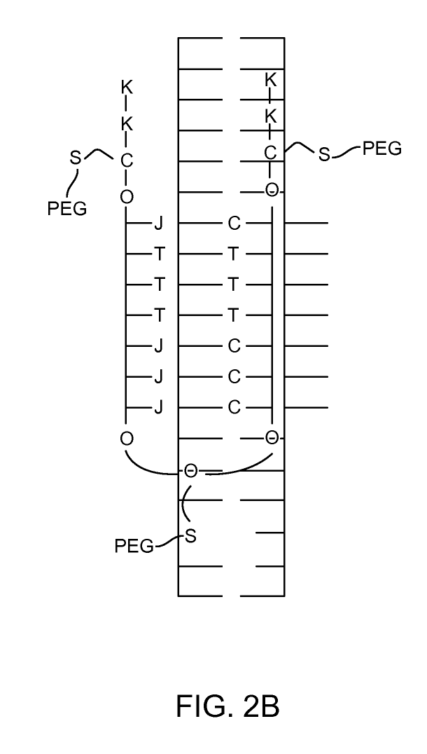

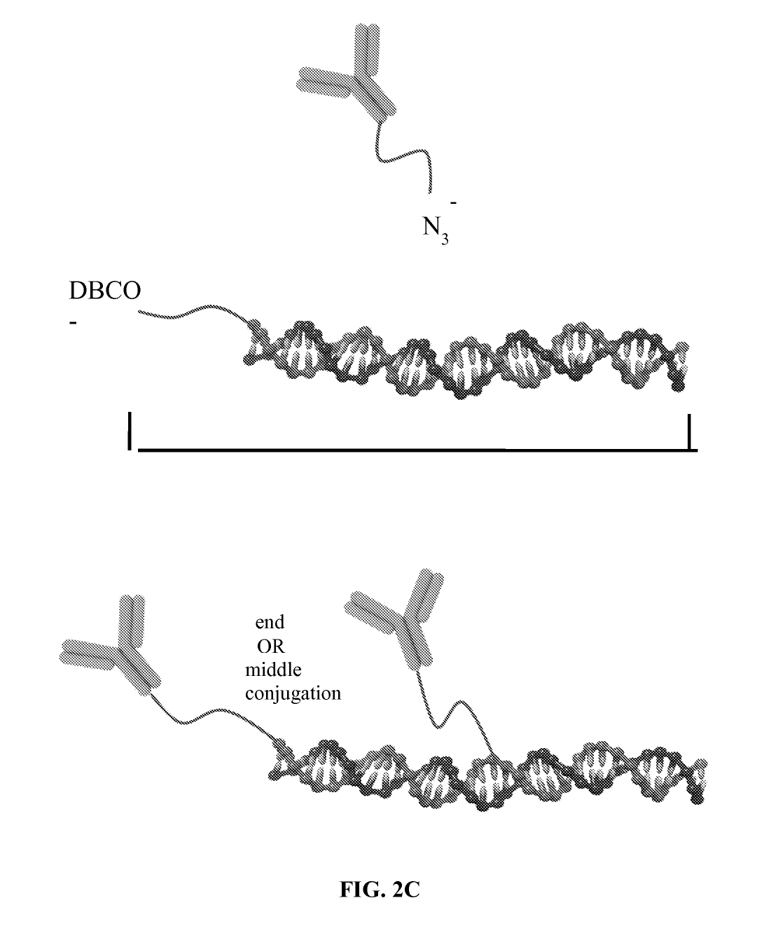

Since the labels can be modified by parameters such as width, length, size, and/or charge, the compositions and methods described herein can be performed with pores of varying size, including larger pores, which are easier and cheaper to manufacture than smaller nanopore devices. For example, FIG. 2 shows a PNA label that has been modified by addition of a detectable tag (in peptides (FIG. 2A), and in polyethylene glycol (FIG. 2B) so as to increase its size, and therefore facilitate detection. This greater size results in a greater change in current flow through the pore, or current impedance, compared to an unlabeled PNA. FIG. 2C demonstrates a method of tagging the scaffold (terminus or within) with a molecule that can act as a label or that can capture analyte.

In an embodiment, therefore, the present technology provides a method for identifying a plurality of label binding domains on a polymer scaffold. The method entails (a) loading a polymer scaffold into a device with a pore that separates and connects two volumes, under conditions that (i) allow a plurality of labels each to specifically bind to one or more of the label binding domains on the polymer scaffold and (ii) allow the polymer scaffold, along with the bound labels, to translocate through the pore from one volume to the other volume, and (b) collecting the electrical signal correlated to the passage of the polymer scaffold through the nanopore. Using the electrical signal, events identifying the translocation of the molecule may be collected and analyzed to identify electrical signals correlated with each label.

An “electrical signal” can include current measurement creating a current signature from the translocation through the pore of one, or alternatively two or more adjacent labels at a time. The identification of multiple labels in an electrical signal may be due to simultaneous location in the nanopore during translocation, or due to sequential location in the nanopore during translocation. When an electrical signal includes only one label, the label needs to be spaced apart from its adjacent labels to avoid the adjacent labels (when all are bound to the polymer scaffold) from interfering with the detection of the label by correlation with the electrical signal.

Due to Brownian motion, the ability to detect a label bound to the polymer scaffold by measuring current impedance changes requires the bound labels to be spaced apart such that each bound label yields a current impedance measurement that is not influenced by neighboring bound labels. Therefore, in some embodiments, the label binding domain is spaced apart from other label binding domains on the polymer scaffold so that only one label is in the pore at a time. For example, if the nanopore is 1 nm in length, the proper separation may be achieved by having label binding domains separated by a distance of at least 1 nm (e.g., approximately 3 nucleotides (nt)). In other words, two adjacent label binding domains are separated by at least 1 nm (or 3 nt) on the polymer scaffold. This separation may be adjusted depending the length of the nanopore used to detect the labels bound to the polymer scaffold. In some aspects, each label binding domain is separated from an adjacent label binding domain on the polymer scaffold by at least 1 nm, 2 nm, 3 nm, 4 nm, 5 nm, 6 nm, 7 nm, 8 nm, 9 nm, 10 nm, 15 nm, 20 nm, 25 nm, 30 nm, 40 nm, 50 nm, 100 nm, 200 nm, 300 nm, 400 nm, or 500 nm. In some embodiments, however, sufficient resolution of labels bound to the polymer scaffold may be achieved without separation of label binding domains on the polymer scaffold.

In some embodiments, adjacent labels may be part of a unique electrical signal used for identification of the label binding domains or bound labels. For instance, labels A and B together may provide one unique electrical signal, whereas the same label B and the next label C can jointly form a different unique electrical signal.

In each of these above scenarios, the nanopore device can be suitably configured to identify each unique electrical signal generated by two or more bound labels without interference from other nearby bound labels. For instance, if the unique electrical signal is generated by two adjacent bound labels as the polymer scaffold passes through the nanopore, the nanopore can be long enough to accommodate both labels.

Because labels can include many molecules along the scaffold, one can construct arbitrarily long sequences of unique labels that encode for arbitrary amounts of information, making it possible to use the entire synthetic structure as a data storage mechanism.

### Molecular Detection

The present disclosure provides methods and systems for molecular detection and quantitation of target analytes in a mixed sample. Further, such detection, quantitation, and measurement can be carried out in a multiplexed manner, greatly increasing its efficiency. Methods and compositions for analyte detection are disclosed in PCT Publication WO/2014/182634, incorporated by reference in its entirety.

In a nanopore experiment, a population of current impedance events is generated. Mathematical modeling is used to pick out our target analytes from background within a degree of confidence. However, a mixed sample, e.g., blood that has not been processed, has a large population of background molecules that produce electrical signals that overlap with those of a target. A high error rate may be introduced by these molecules, affecting the reliability of the nanopore to detect target analytes. One mechanism to improve reliability, as disclosed herein, is too attach a label or a sequence of labels to a polymer scaffold to provide a unique electrical signal that can be used to identify the presence and/or identity of a polymer scaffold that has translocated through a nanopore. These polymer scaffolds contain fusion molecule binding domains to bind fusion molecules which bind directly, or through an intermediary, to a target analyte. This detection provides a unique electrical signal upon translocation through the nanopore that can be further discriminated from background molecules by identification of a label or sequence of labels attached to the polymer scaffold. Therefore, provided are improved methods and compositions for detecting target analytes in a bulk sample using a nanopore.

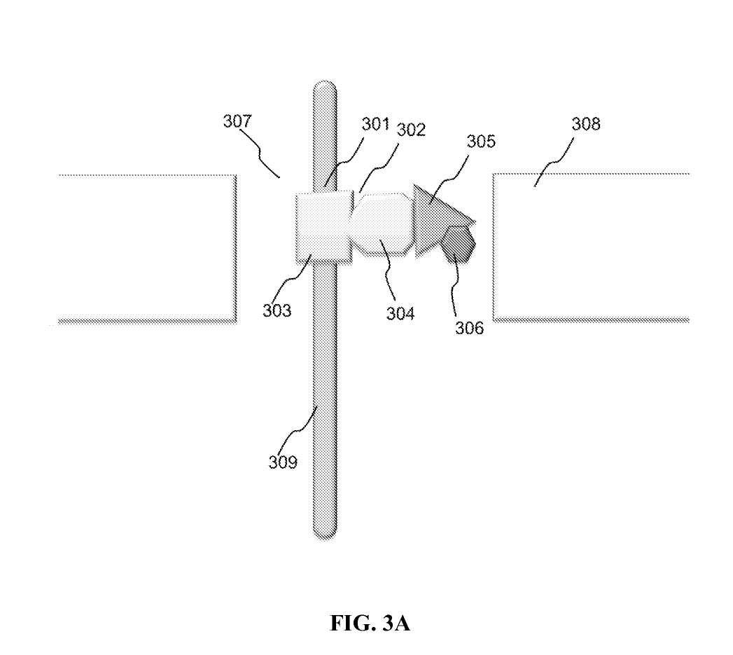

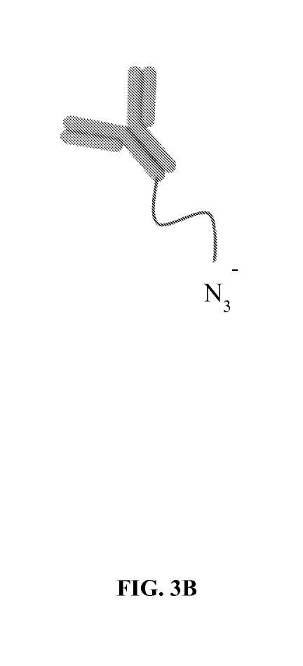

FIG. 3A provides an illustration of an embodiment of the disclosed methods and systems. More specifically, the system includes a ligand comprising an analyte binding moiety 304 that is capable of binding to a target analyte 305 to be detected or quantitated. The ligand 304 can be part of, or be linked to, a scaffold binding moiety (i.e., a “scaffold binding domain”) 303 that is capable of binding to a specific binding motif or fusion molecule binding domain (e.g., a DNA sequence) 301 on a polymer scaffold 309. The ligand, shown in FIG. 3B, can be directly chemically coupled to the scaffold through the binding moiety (as described in [0046] and FIG. 2C). Together, the ligand 304 and the scaffold binding moiety 303 form a fusion molecule 302. In various embodiments, both components of the fusion molecule 302 (i.e., both the ligand 304 and the scaffold binding moiety 303) bind to their respective targets (e.g., target analyte 305 and fusion molecule binding domain 301, respectively) with high affinity and specificity.

Therefore, if all are present in a solution, the fusion molecule 302 binds, on one end, to a polymer scaffold (or simply, “polymer”) 309 through the specific recognition and binding between the fusion molecule binding domain 301 and the scaffold binding moiety 303, and on the other end, to the target analyte 305 by virtue of the interaction between the analyte binding moiety on the ligand 304 and the target analyte 305. Such bindings cause the formation of a complex (i.e., a formed complex) that includes the polymer scaffold 309, the fusion molecule 302 and the target analyte 305.

However, detection of a target analyte in a bulk sample may still be difficult due to the presence of background molecules which provide a variety of current impedance signatures which may be hard to distinguish of the formed complex in a nanopore. Therefore, the attachment of labels to the polymer scaffold to provide a unique electrical signal that is part of the event used to detect the analyte-fusion molecule complex may be used to identify the polymer scaffold as causing the event. Therefore, electrical signals that are part of the event caused by the polymer scaffold translocating through the nanopore can be distinguished from background molecules in an unfiltered bulk sample, such as whole blood. This innovative method of detection and polymer scaffold composition provides a quick and effective means of identifying target analytes in an unfiltered sample, while reducing error of false positive or false negatives in detection.

The formed complex can be detected using a device 308 that includes a nanopore (or simply, pore) 307, and a sensor. The pore 307 is a nano-scale or micro-scale opening in a structure separating two volumes. The sensor is configured to identify objects passing through the pore 307. For example, in some embodiments, the sensor identifies objects passing through the pore 307 by detecting a change in a measurable parameter, wherein the change is indicative of an object passing through the pore 307. This device is referred throughout as a “nanopore device.” In some embodiments, the nanopore device 308 includes electrodes connected to power sources, for moving the polymer scaffold 309 from one volume to another, across the pore 307. As the polymer scaffold 309 can be charged or be modified to contain charges. By generating a potential or voltage across the pore 307 the movement of the polymer scaffold 309 is facilitated and controlled. In certain embodiments, the sensor comprises a pair of electrodes, which are configured both as a sensor to detect the passage of objects through the nanopore by reading current, and to provide a voltage, across the pore 307. In certain embodiments, a voltage-clamp or a patch-clamp is used to simultaneously supply a voltage across the pore and measure the current through the pore.

When a sample that includes the formed complex is in the nanopore device 308, the nanopore device 308 can be configured to pass the formed complex including the polymer scaffold 309 through the pore 307. When the fusion molecule binding domain 301 is within the pore or adjacent to the pore 307, the binding status of the fusion molecule binding domain 301 can be detected by the sensor through current impedance or equivalent electrical signature.

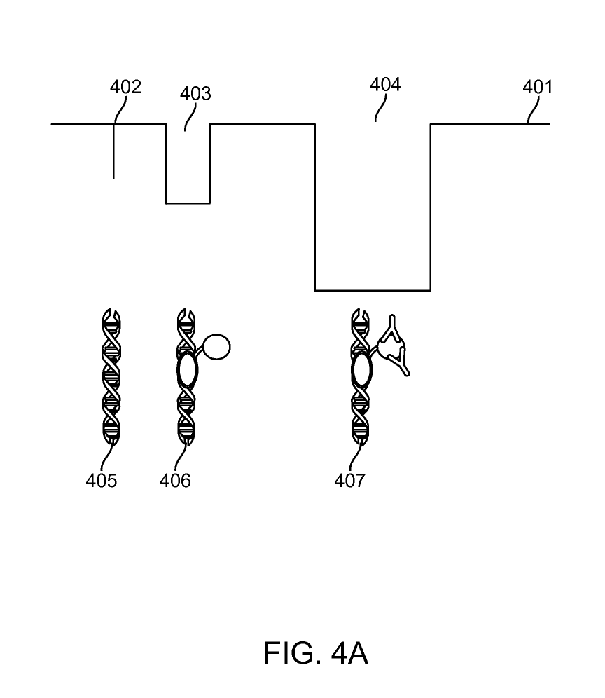

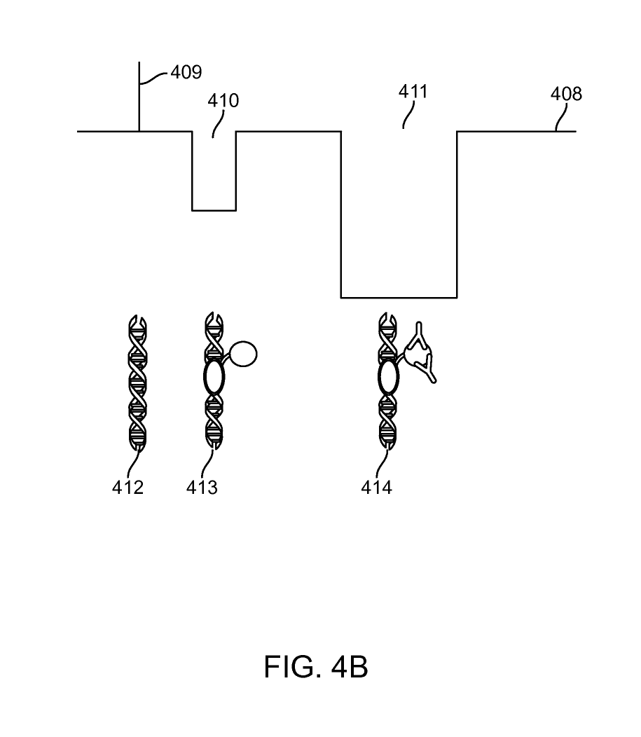

The “binding status” of a fusion molecule binding domain, as used herein, refers to whether the fusion molecule binding domain is bound to a fusion molecule with a corresponding scaffold binding domain, and whether the fusion molecule is also bound to a target analyte. Essentially, the binding status can be one of three potential statuses: (i) the fusion molecule binding domain is free and not bound to a fusion molecule (see 405 in FIG. 4); (ii) the fusion molecule binding domain is bound to a fusion molecule that does not bind to a target analyte (see 406 in FIG. 4); or (iii) the fusion molecule binding domain is bound to a fusion molecule that is bound to a target analyte (see 407 in FIG. 4).

Detection of the binding status of a fusion molecule binding domain can be carried out by various methods. In one aspect, by virtue of the different sizes of molecules attached to the binding domain at each status, when the binding domain passes through the pore, the electrical signal will correlate to the binding status. In one aspect, as shown in FIG. 4A, with a positive voltage applied and KCI concentrations greater than 0.4 M in the experiment buffer, the measured current signals 401, when 405, 406, and 407 pass through the pore, are signals 402, 403, and 404, respectively. All three event types are subjected to current attenuation when KCI concentrations are greater than 0.4 M, causing a reduction in the positive current flow. The three signals 402, 403, and 404 can be differentiated from one another by the amount of the current shift (height) and/or the duration of the current shift (width), or by any other feature in the signal that differentiates the three event types. It can also be that 404 is commonly different than 402 and 403, but that 402 and 403 are not commonly different from each other, in which case, robust detection of the biomarker bound to the passing molecule can still be accomplished. In another aspect, as shown in FIG. 4B, with a positive voltage applied and KCI concentrations less than 0.4M in the experiment buffer, the measured current signals 408, when 412, 413, and 414 pass through the pore, are signals 409, 410, and 411, respectively. Passage of dsDNA alone causes current enhancement events (409) at KCI concentrations less than 0.4 M. This was shown in the published research by Smeets, Ralph M M, et al. “Salt dependence of ion transport and DNA translocation through solid-state nanopores.” Nano Letters 6.1 (2006): 89-95. Hence, the signal 409 can be differentiated from 410 and 411 by the event amplitude direction (polarity) relative to the open channel baseline current level (408), in addition to the three signals commonly having different amounts of the current shift (height) and/or the duration of the current shift (width), or by any other feature in the signal that differentiates the three event types. In another aspect, as shown in FIG. 4C, with a negative voltage applied and KCI concentrations less than 0.4 M in the experiment buffer, the negative measured current signals 415, when 419, 420, and 421 pass through the pore, are signals 416, 417, and 418, respectively. Compared to signals 409, 410, and 411 with a positive voltage, the signals 416, 417, and 418 have the opposite polarity since the applied voltage has the opposite (negative) polarity. In all aspects of the FIG. 4 embodiments, the sensor comprises electrodes, which are connected to power sources and can detect the current. Either one or both of the electrodes, therefore, serve as a “sensor.” In this embodiment, a voltage-clamp or a patch-clamp is used to simultaneously supply a voltage across the pore and measure the current through the pore.

In some aspects, an agent 306 as shown in FIG. 3 is added to the complex to aid detection. This agent is capable of binding to the target analyte or the ligand/target analyte complex. In one aspect, the agent includes a charge, either negative or positive, to facilitate detection. In another aspect, the agent adds size to facilitate detection. In another aspect, the agent includes a detectable label, such as a fluorophore.

In this context, an identification of status (iii) indicates that a polymer scaffold-fusion molecule-target analyte complex has formed. In other words, the target analyte is detected.

### Larger Analyte Detection

The present disclosure also provides, in some aspects, methods and systems for detecting, quantitating, and measuring target analytes such as proteins, protein aggregates, oligomers, or protein/DNA complexes, or cells and microorganisms, including viruses, bacteria, and cellular aggregates.

In some aspects, the pore within the structure that separates the device into two volumes has a size that allows larger analytes, such as viruses, bacteria, cells, or cellular aggregates, to pass through. A fusion molecule having a ligand with an analyte binding moiety capable of binding to a larger target analyte to be detected or quantitated can be included in the solution in the nanopore device such that the ligand can bind to the unique target analyte and the polymer scaffold through a fusion molecule, generating a formed complex with the target analyte. Many such analytes have unique markers on their surfaces that can be specifically recognized by an analyte binding moiety on the ligand. For instance, tumor cells can have tumor antigens expressed on the cell surface, and bacterial cells can have endotoxins attached on the cell membrane.

When the formed complex in a solution loaded into the nanopore device is moved along with the polymer scaffold to pass through the pore, the binding status of the fusion molecule to the target analyte within or adjacent to the pore can be detected such that the analytes bound to the ligands can be identified using methods similar to the molecular detection methods described elsewhere in the disclosure.

### Multiplexing

In some aspects, rather than including multiple fusion molecule binding domains of the same kind as described above, a polymer scaffold can include multiple types of fusion molecule binding domains, each having different corresponding binding domains. In such embodiments, a sample can include multiple types of fusion molecules, each type including one of the different corresponding binding domains and a ligand for a different target analyte.

An additional method of multiplexing includes assaying a collection of different scaffold molecules during a test, with each different scaffold associating with different fusion molecule(s). To determine what target analytes are in solution, scaffolds of the same type are labeled such that the sensor can identify what fusion molecule will bind to that particular scaffold. This can be accomplished, for example, by barcoding each type of scaffold with polyethylene glycol molecules of varying lengths or sizes.

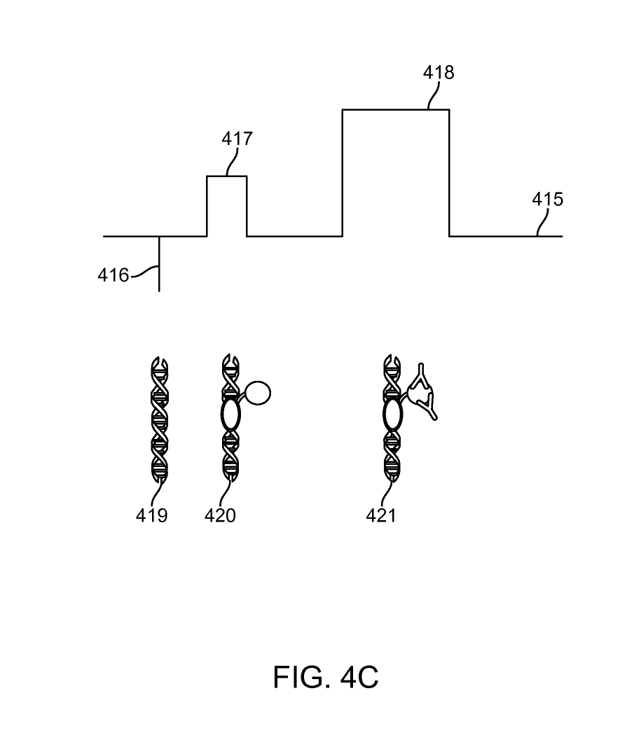

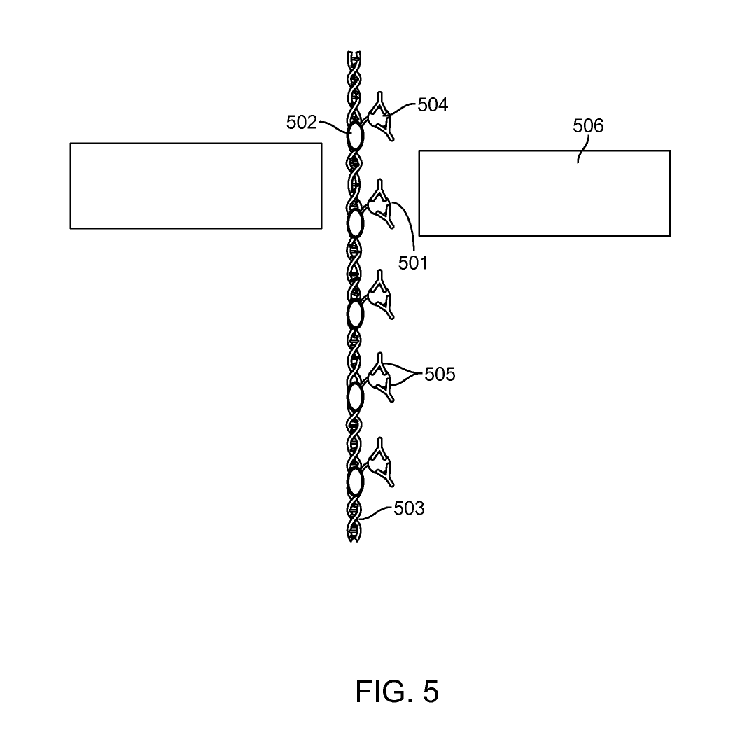

With such a setting, a single polymer scaffold can be used to detect multiple types of target analytes, including target molecules, target microorganisms (e.g. bacterium or virus), or target cells (e.g. circulating tumor cells). FIG. 5 illustrates such a method. Here, a double-stranded DNA 503 is used as the polymer scaffold, the double-stranded DNA 503 including multiple fusion molecule binding domains: two copies of a first fusion molecule binding domain 504, two copies of a second fusion molecule binding domain 505, and one copy of a third fusion molecule binding domain 506.

In some embodiments, the multiplexing polymer scaffold also comprises at least one label bound to a label binding domain on the polymer scaffold. In this manner, an electrical signal provided by the label—polymer scaffold complex can identify the polymer scaffold in an event. Thus, individual electrical signals attributed to polymer scaffold—fusion molecule complexes can be more easily detected and analyzed to determine the presence of an analyte based on the electrical signal.

When the DNA passes through a nanopore device 507 that has two coaxial pores, the binding status of each of the fusion molecule binding domains is detected. Each fusion molecule binding domain 504 bind to a corresponding target analyte. In one embodiment, electrical signals arising from unique bound fusion molecules 504 are distinguishable from other fusion molecule analyte complexes, and thus can be used for multiplexed detection of analytes on a single scaffold. In certain embodiments, the electrical signals from fusion molecules can be read in sequence and their identity determined by their relative position. Whether or not the fusion molecule is bound to an analyte can be detected as the DNA passes through a nanopore device.

This way, with a single polymer scaffold and a single nanopore device, the present technology can simultaneously detect multiple different target analytes. Further, by determining how many copies of fusion molecule binding domains are bound to the target analytes, and by tuning conditions that impact the bindings, the system can obtain more detailed binding dynamic information.

### Polymer Scaffold

A polymer scaffold suitable for use in the present technology is a scaffold that can be loaded into a nanopore device and passed through the pore from one end to the other.

Non-limiting examples of polymer scaffolds include nucleic acids, such as deoxyribonucleic acid (DNA), ribonucleic acid (RNA), or peptide nucleic acid (PNA), dendrimers, and linearized proteins or peptides. In some aspects, the DNA or RNA can be single-stranded or double-stranded, or can be a DNA/RNA hybrid molecule.

In certain embodiments, double stranded DNA is used as a polymer scaffold. There are several advantages of dsDNA over ssDNA as a polymer scaffold. In general, non-specific interactions and unpredictable secondary structure formation are more prevalent in ssDNA, making dsDNA more suitable for generating reproducible electrical signals in a nanopore device. Also, ssDNA elastic response is more complex than dsDNA, and the properties of ssDNA are less well known than for dsDNA. Therefore, many embodiments of the invention are engineered to encompass dsDNA as a polymer scaffold, including several of the labels and fusion molecules used herein.

In one aspect, the polymer scaffold is synthetic or chemically modified. Chemical modification can help to stabilize the polymer scaffold, add charges to the polymer scaffold to increase mobility, maintain linearity, or add or modify the binding specificity, or add chemically reactive sites to which labels or ligands can be tethered. In some aspects, the chemical modification is acetylation, methylation, summolation, oxidation, phosphorylation, glycosylation, thiolation, addition of azides, or alkynes or activated alkynes (DBCO-alkyne), or the addition of biotin.

In some aspects, the polymer scaffold is electrically charged. DNA, RNA, PNA and proteins are typically charged under physiological conditions. Such polymer scaffolds can be further modified to increase or decrease the carried charge. Other polymer scaffolds can be modified to introduce charges. Charges on the polymer scaffold can be useful for driving the polymer scaffold to pass through the pore of a nanopore device. For instance, a charged polymer scaffold can move across the pore by virtue of an application of voltage across the pore.

In some aspects, when charges are introduced to the polymer scaffold, the charges can be added at the ends of the polymer scaffold. In some aspects, the charges are spread over the polymer scaffold.

In an embodiment, each unit of the charged polymer scaffold is charged at the pH selected. In another embodiment, the charged polymer scaffold includes sufficient charged units to be pulled into and through the pore by electrostatic forces. For example, a peptide containing sufficient entities can be charged at a selected pH (lysine, aspartic acid, glutamic acid, etc.) so as to be used in the devices and methods described herein. Likewise, a co-polymer comprising methacrylic acid and ethylene is a charged polymer for the purposes of this invention if there is sufficient charged carboxylate groups of the methacrylic acid residue to be used in the devices and methods described herein. In an embodiment, the charged polymer scaffold includes one or more charged units at or close to one terminus of the polymer scaffold. In another embodiment, the charged polymer scaffold includes one or more charged units at or close to both termini of the polymer scaffold. One co-polymer example is a DNA wrapped around protein (e.g. DNA/nucleosome). Another example of a co-polymer is a linearized protein conjugated to DNA at the N- and C-terminus

The much-improved polymer scaffold decoding technology as provided above makes it practical to use polymer scaffolds for data storage, which is also within the scope of the present disclosure. For instance, using the codes (A, B, C and D) illustrated in FIG. 1, a polynucleotide can be synthesized, including label binding domains for labels A, B, C and/or D. Such label binding domains can be detected by a nanopore device as presently described, through binding to the corresponding labels. In other embodiments, A, B, C, and D themselves are labels, insofar as they generate detectably different signals when passing through the nanopore. Therefore, the composition and sequence of the polynucleotide in terms of the label binding domains constitute an information storage, and A, B, C and D represent the code of the storage.

Using polymer scaffolds for data storage, it is contemplated there are many advantages over conventional computer memory technologies, which are bound to a binary number system (0's and 1's) due to the fact that data is stored using electronic gates, which can be in only one of two states (on and off) for each location in the memory unit. The presently disclosed technology can accommodate an arbitrarily large number of different labels in the same location, as described below; hence the variation in each code is much greater than the 1's and 0's of a binary system. Accordingly, the data capacity of each unit is much greater. Further, decoding of the data can be faster, given that nanopore-based label detection can be multiplexed in parallel, where hundreds, thousands or each millions of nanopores on a single membrane.

Thus, in certain embodiments, the present disclosure provides a polymer scaffold-based data storage device and methods for encoding and decoding the data in the device. The polymer scaffold can be synthesized to include label binding domains which serve as codes for the data. Before or during reading the data, the polymer scaffold is placed in contact with the labels under conditions where the labels can bind to the label binding domains. The polymer scaffold that is bound to the labels can then be subjected to label detection by a nanopore device. Finally, the detected labels can be compiled to represent the data.

In some aspects, the labels can be permanently linked to the polymer scaffold. For example this can be done by cross-linking the labels to the scaffold using formaldehyde if the labels are proteins. In another aspect, chemical coupling can be used to link the label to the scaffold.

### Probe Binding Domains on the Scaffold

For nucleic acids and polypeptides such as the polymer scaffold, a probe (e.g., a label or fusion molecule) binding domain can be a nucleotide or peptide sequence that is recognizable by a scaffold binding domain on the probe. In some embodiments, the probe binding domain is a peptide sequence forming a functional portion of a protein, although the binding domain does not have to be a protein. For nucleic acids, for instance, there are proteins that specifically recognize and bind to sequences (motifs) such as promoters, enhancers, thymine-thymine dimers, and certain secondary structures such as bent nucleotide and sequences with single-strand breakage.

In some aspects, the probe binding domain includes a chemical modification that causes or facilitates recognition and binding by a polymer scaffold binding domain. For example, methylated DNA sequences can be recognized by transcription factors, DNA methyltransferases or methylation repair enzymes. In other embodiments, biotin may be incorporated into, and recognized by, avidin family members. In such embodiments, biotin forms the probe binding domain and avidin or an avidin family member is the polymer scaffold binding domain on the probe. Due to their binding complementarity, probe binding domains and polymer scaffold domains may be reversed so that the probe binding domain becomes the polymer scaffold binding domain, and vice versa.

Molecules, in particular proteins, that are capable of specifically recognizing nucleotide binding motifs are known in the art. For instance, protein domains such as helix-turn-helix, a zinc finger, a leucine zipper, a winged helix, a winged helix turn helix, a helix-loop-helix and an HMG-box, are known to be able to bind to nucleotide sequences.

In some aspects, the probe binding domains can be locked nucleic acids (LNAs), bridged nucleic acids (BNA), Protein Nucleic Acids of all types (e.g. bisPNAs, gamma-PNAs), transcription activator-like effector nucleases (TALENs), clustered regularly interspaced short palindromic repeats (CRISPRs), or aptamers (e.g., DNA, RNA, protein, or combinations thereof).

In some aspects, the probe binding domains are one or more of DNA binding proteins (e.g., zinc finger proteins), antibody fragments (Fab), chemically synthesized binders (e.g., PNA, LNA, TALENS, or CRISPR), or a chemical modification (i.e., reactive moieties) in the synthetic polymer scaffold (e.g., thiolate, biotin, amines, carboxylates).

In some embodiments, the polymer scaffold includes a sequence of label binding domains which are used to encode information in the polymer scaffold. In other embodiments, the polymer scaffold also includes a fusion molecule binding domain for analyte detection, in combination with at least one label binding domain for scaffold identification. In some embodiments, the polymer scaffold can include a plurality of unique fusion molecule binding domains for multiplexed analyte detection on a single polymer scaffold.

### Labels

In some embodiments, the label includes a protein that specifically recognizes and binds a specific label binding domain on the polymer scaffold. For nucleic acids and polypeptides as the polymer scaffold, a label binding domain can be a nucleotide or peptide sequence that is recognizable by a binding protein, which is typically a functional portion of a protein. For nucleic acids, for instance, there are proteins that specifically recognize and bind to sequences (motifs) such as promoters, enhancers, thymine-thymine dimers, and certain secondary structures such as bent nucleotide and sequences with single-strand breakage.

In some aspects, the label includes a chemical modification that causes or facilitates recognition and binding by a label binding domain. For example, methylated DNA sequences can be recognized by transcription factors, DNA methyltransferases or methylation repair enzymes.

Molecules, in particular proteins, that are capable of specifically recognizing nucleotide binding domains are known in the art. For instance, protein domains such as helix-turn-helix, a zinc finger, a leucine zipper, a winged helix, a winged helix turn helix, a helix-loop-helix and an HMG-box, are known to be able to bind to nucleotide sequences.

Any molecule that specifically binds to a label binding domain on a polymer scaffold, which can be characterized by the sequence or structure, can be a label. Examples of label molecules include a peptide, a nucleic acid, TALENS, CRISPR, LNA, a PNA (protein nucleic acid), bis-PNA, gamma-PNA, a PNA-conjugate that increases size or charge of PNA, or any other PNA derived polymer, and a chemical compound, e.g. polyethylene glycol of various lengths.

A PNA is a synthetic form of nucleic acid which lacks a net electrical charge along its protein-like backbone. PNAs have found a number of applications in vitro, as well as in vivo to tag specific genomic sequences. In one aspect, at least one label is a bis-PNA. A bis-PNA molecule is made up of two PNA oligomers connected by a flexible linker. A few lysine residues are often added at their termini to improve association kinetics to dsDNA. It can spontaneously target dsDNA molecules with high affinity and sequence-specificity, relying on the simultaneous formation of Watson-Crick and Hoogsteen base-pairs. In other embodiments, the PNA can have certain modifications, such as in pseudo-complementary PNA (i.e., pcPNA) and gamma-PNA (i.e., γ-PNA). The synthesis of PNAs are well known in the art.

Generally, a bis-PNA is comprised of homopyrimidines or homopurines, and its binding of dsDNA generally requires a PNA/DNA triplex formation. This essentially limits the target regions for hybridization on the dsDNA to homopurine homopyrimidine stretches. In order to avoid the sequence limitations associated with PNAs such as bis-PNAs, so as to be able to target essentially any mixed DNA sequence, other modified PNA labels can be used.

In some aspects, the at least one label is a γ-PNA. γ-PNA has a simple modification in a peptide-like backbone, specifically at the γ-position of the N-(2-aminoethyl)glycine backbone, thus generating a chiral center (Rapireddy S., et al., 2007. J. Am. Chem. Soc., 129:15596-600; He G, et al., 2009, J. Am. Chem. Soc., 131:12088-90; Chema V, et al., 2008, Chembiochem 9:2388-91; Dragulescu-Andrasi, A., et al., 2006, J. Am. Chem. Soc., 128:10258-10267). Unlike bis-PNA, γ-PNA can bind to dsDNA without sequence limitation, leaving one of the two DNA strands accessible for further hybridization.

In some aspects, the function of the label is to hybridize to the polymer scaffold by complement base pairing to form a stable complex. That complex has sufficiently large cross-section surface area to produce a detectable change or contrast in signal amplitude over that of the background, which is the mean or average signal amplitude corresponding to sections of non-label-bound target-bearing polymer scaffold.

The stability of the complex is important in order for it to be detected by a nanopore device. The complex's stability must be maintained throughout the period that the target-bearing polymer scaffold is being translocated through the nanopore. If the complex is weak, or unstable, the complex can fall apart and will not be detected as the target-bearing polymer scaffold threads through the nanopores.

In some aspects, the labels can be permanently linked to the polymer scaffold. For example this can be done by cross-linking the labels to the scaffold using formaldehyde if the labels are proteins. In another aspect, chemical coupling can be used to link the label to the scaffold.

The size of the complex including the polymer scaffold and the label has to have sufficient properties, e.g., size and charge, to generate a detectable electrical signal when the complex threads through the nanopore which deviates from the background noise. In some embodiments, this may be performed by adding a detectable tag to a label comprising a polymer scaffold binding domain. This detectable tag may be modified by its width, length, size, or charge to affect the electrical signal generated by measuring current impedance as the label comprising a detectable tag and bound to a polymer scaffold translocates through the nanopore. An example of the use of labels attached to detectable tags is shown in FIG. 1, where labels A, B, C, and D each have a unique detectable tag to generate a distinguishable electrical signal to allow identifications of the labels, and therefore the label binding sites, as the polymer scaffold translocates through the nanopore.

FIG. 2 shows a PNA label that has been modified by addition of a detectable tag so as to increase its size, and therefore facilitate detection. Specifically, this label, which binds to the target DNA sequence by complementary base pairing between the bases on the PNA molecule (204) and the bases in the target DNA, has cysteine residues incorporated into the backbone (201 dotted line box), which provide a free thiol chemical handle for conjugation to a detectable tag. Here, the cysteine is attached to a peptide (203) through a maleimide linker (202 dotted line box). The peptide acts as a detectable tag, providing a means to better detect whether the label is bound to its target sequence upon translocation through the nanopore, since the label/peptide gives an increase to the label size. This greater size results in a greater change in current flow through the pore, or current impedance, compared to an unlabeled PNA.

In a particular embodiment, a label is a PNA conjugated to a detectable tag, in which the PNA portion specifically recognizes a nucleotide sequence, and the detectable tag increases the size/shape/charge differences between different PNA conjugates.

In some aspects, to increase the contrast in the change between the label-bound polymer scaffold complex and other molecules present in the sample, modification can be made to the pseudo-peptide backbone to change the overall charge of the label (e.g., PNA) to increase the contrast. Selection of more charged amino acids instead of non-polar amino acids can serve to increase the charge of PNA. In addition, smaller detectable tags, such as molecules, proteins, peptides, or polymers (e.g., PEG) can be conjugated to the pseudo-peptide backbone to enhance the bulk or cross-sectional surface area of the label and target-bearing polymer scaffold complex. Enhanced bulk serves to enhance the signal amplitude contrast so that any differential signal resulting from the increased bulk can be easily detected. Small molecules, such as organic molecules, proteins, or peptides, can be conjugated to the pseudo-peptide backbone. These molecules include, but are not limited to, nanometer-sized gold particles (e.g. 3 nm), quantum dots, polyethylene glycol (PEG), polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and trypsin inhibitors. Methods of conjugation of molecules are well known in the art, e.g. in U.S. Pat. Nos. 5,180,816, 6,423,685, 6,706,252, 6,884,780, and 7,022,673, which are hereby incorporated by reference in their entirety. Examples of some conjugating agents include, but are not limited to, ethylenediaminetetraacetic acid (EDTA), diethylenetriaminopentaacetic acid (DTPA), ethyleneglycol-0,0′-bis(2-aminoethyl)-N,N,N′,N′-tetraacetic acid (EGTA), N,N′-bis(hydroxybenzyl)ethylenediamine-N,N′-diacetic acid (HBED), triethylenetetraminehexaacetic acid (TTHA), 1,4,7,10-tetra-azacyclododecane-N,N′,N″,N′″-tetraacetic acid (DOTA), 1,4,7,10-tetraazacyclotridecane-1,4,7,10-tetraacetic acid (TITRA), 1,4,8,11-tetraazacyclotetradecane-N,N′,N″,N′″-tetraacetic acid (TETA), and 1,4,8,11-tetraazacyclotetradecane (TETRA).

In some aspects, the label needs not entirely hybridize to the target-bearing polymer scaffold. It can be sufficient that a portion of the label binds to the target-bearing polymer scaffold. In some aspects, at least 50% of the label binds to the target-bearing polymer scaffold. In some aspects, at least 5%, at least 10%, at least 15%, at least 25%, at least 30%, at least 35%, at least 40%, or at least 45% of the label binds to the target-bearing polymer scaffold.

Different reactive moieties may be incorporated into the labels to provide chemical handles to which labels may be conjugated to serve as detectable tags. Examples of reactive moieties, which maybe included in the scaffold itself or probe (such as PNA), include, but are not limited to, primary amines, carboxylic acids, ketones, amides, aldehydes, boronic acids, hydrazones, thiols, maleimides, alcohols, and hydroxyl groups.

A common method for incorporating the chemical handles is to include a specific amino acid into the backbone of the label. Examples include, but are not limited to, cysteines (provide thiolates), lysines (provide free amines), threonine (provides hydroxyl), glutamate and aspartate (provides carboxylic acids). Examples of this are detectable tags that add size, charge, or fluorescence to the label.

Different types of labels can be added using the reactive moieties. These include labels that: 1) increase the size of the label, e.g. biotin/streptavidin, peptide, nucleic acid; 2) change the charge of the label, e.g. a charged peptide (6×HIS), or protein (charybdotoxin); and 3) change or add fluorescence to the label, e.g. common fluorophores, FITC, Rhodamine, Cy3, Cy5.

The labels may be detected by methods known in the art as an alternative to the use of current impedance. Useful labels include, e.g., fluorescent dyes (e.g., Cy5®, Cy3®, FITC, rhodamine, lanthamide phosphors, Texas red), 32P, 35S, 3H, 14C, 125I, 131I, electron-dense reagents (e.g., gold), enzymes as commonly used in an ELISA (e.g., horseradish peroxidase, beta-galactosidase, luciferase, alkaline phosphatase), colorimetric labels (e.g., colloidal gold), magnetic labels (e.g., Dynabeads™), biotin, dioxigenin, or haptens and proteins for which antisera or monoclonal antibodies are available. Other labels include labels or oligonucleotides capable of forming a complex with the corresponding receptor or oligonucleotide complement, respectively. The label can be directly incorporated into the nucleic acid to be detected, or it can be attached to a label (e.g., an oligonucleotide) or antibody that hybridizes or binds to the nucleic acid to be detected.

In some aspects, the label is a fluorophore. The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

Suitable fluorescent moieties include the following fluorophores known in the art: 4-acetamido-4′-isothiocyanatostilbene-2,2′disulfonic acid acridine and derivatives: acridine, acridine isothiocyanate, Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS), 4-amino-N-(3-vinylsulfonyl) phenyl]naphthalimide-3,5 disulfonate (Lucifer Yellow VS), N-(4-anilino-1-naphthyl) maleimide, anthranilamide, Black Hole Quencher™ (BHQ™) dyes (biosearch Technologies), BODIPY® R-6G, BOPIPY® 530/550, BODIPY® FL Brilliant Yellow; coumarin and derivatives: coumarin, 7-amino-4-methylcoumarin (AMC, Coumarin 120); 7-amino-4-trifluoromethylcouluarin (Coumarin 151), Cy2®, Cy3®, Cy3.5®, Cy5®, Cy5.5®; Cyanosine 4′,6-diaminidino-2-phenylindole (DAPI) 5′,5″-dibromopyrogallol sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid 5 [dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC), Eclipse™ (Epoch Biosciences Inc.); eosin and derivatives: eosin, eosin isothiocyanate; erythrosin and derivatives: erythrosin B, erythrosin isothiocyanate, ethidium fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET), fluorescamine, IR144, IR1446, Malachite Green isothiocyanate, 4-methylumbelliferone, ortho cresolphthalein, nitrotyrosine, pararosaniline, Phenol Red, B-phycoerythrin, R-phycoerythrin, o-phthaldialdehyde, Oregon Green®, propidium iodide; pyrene and derivatives: pyrene, pyrene butyrate, succinimidyl 1-pyrene butyrate, QSY® 7, QSY® 9, QSY® 21, QSY® 35 (Molecular Probes), Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives.

Other fluorescent nucleotide analogs can be used, see, e.g., Jameson et al., 278 Meth. Enzymol. 363-390 (1997); Zhu et al., 22 Nucl. Acids Res. 3418-3422 (1994). U.S. Pat. Nos. 5,652,099 and 6,268,132 also describe nucleoside analogs for incorporation into nucleic acids, e.g., DNA and/or RNA, or oligonucleotides, via either enzymatic or chemical synthesis to produce fluorescent oligonucleotides. U.S. Pat. No. 5,135,717 describes phthalocyanine and tetrabenztriazaporphyrin reagents for use as fluorescent labels.

The labels can be incorporated into, associated with, or conjugated to, a nucleic acid. Labels can be attached by spacer arms of various lengths to reduce potential steric hindrance or impact on other useful or desired properties. See, e.g., Mansfield, 9 Mol. Cell. Probes 145-156 (1995).

The labels can be incorporated into nucleic acids by covalent or non-covalent means, e.g., by transcription, such as by random-primer labeling using Klenow polymerase, or nick translation, or amplification, or equivalent, as is known in the art. For example, a nucleotide base is conjugated to a detectable moiety, such as a fluorescent dye, e.g., Cy3® or Cy5®, and then incorporated into genomic nucleic acids during nucleic acid synthesis or amplification. Nucleic acids can thereby be labeled when synthesized using Cy3®- or Cy5®-dCTP conjugates mixed with unlabeled dCTP.

Nucleic acid labels can be modified by using PCR or nick translation in the presence of labeled precursor nucleotides, for example. Modified nucleotides synthesized by coupling allylamine-dUTP to the succinimidyl-ester derivatives of the fluorescent dyes or haptens (e.g., biotin or digoxigenin) can be used; this method allows custom preparation of most common fluorescent nucleotides, see, e.g., Henegariu et al., Nat. Biotechnol. 18:345-348 (2000).

Nucleic acid labels may be labeled by non-covalent means known in the art. For example, Kreatech Biotechnology's Universal Linkage System® (ULS®) provides a non-enzymatic labeling technology, wherein a platinum group forms a coordinative bond with DNA, RNA or nucleotides by binding to the N7 position of guanosine. This technology may also be used to label proteins by binding to nitrogen and sulfur containing side chains of amino acids. See, e.g., U.S. Pat. Nos. 5,580,990; 5,714,327; and 5,985,566; and European Patent No. 0539466.

### Fusion Molecule

A “fusion molecule” is intended to mean a molecule or complex that contains two functional regions, a polymer scaffold binding domain and a ligand comprising an analyte binding moiety. The polymer scaffold binding domain is capable of binding to a fusion molecule binding domain on a polymer scaffold, and the ligand is capable of binding to a target analyte.

In some aspects, the fusion molecule is prepared by linking the two regions with a bond or force. Such a bond and force can be, for instance, a covalent bond, a hydrogen bond, an ionic bond, a metallic bond, van der Walls force, hydrophobic interaction, or planar stacking interaction.

In some aspects, the fusion molecule, such as a fusion protein, can be expressed as a single molecule from a recombinant coding nucleotide. In some aspects, the fusion molecule is a natural molecule having a polymer scaffold binding domain and a ligand suitable for use in the present technology.

Many options exist for connecting the polymer scaffold binding domain with the ligand to form the fusion molecule. For example, the components may be connected via chemical coupling through functionalized linkers such as free amine, carboxylate coupling, thiolate, hydrazide, or azide (click) chemistry or the polymer scaffold binding domain and the ligand may form one continuous transcript.

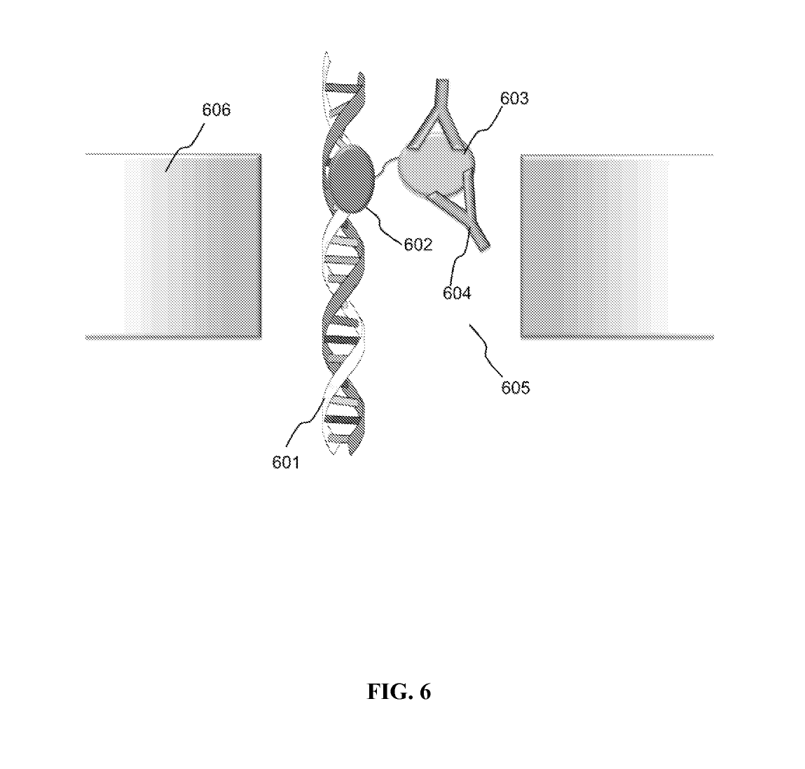

FIG. 6 illustrates a more specific embodiment of the system shown in FIG. 3. In FIG. 6, the fusion molecule is a chimeric protein that includes a zinc finger protein or domain 602 and a human immunodeficiency virus (HIV) envelop protein 603. The zinc finger protein 602 has polymer scaffold binding domain that can bind to a suitable nucleotide sequence on the polymer scaffold, a double-stranded DNA 601; the HIV envelop protein 603 is a ligand with an analyte binding moiety that can bind to an anti-HIV antibody 604 which can be present in a biological sample (e.g., a blood sample from a patient) for detection.

When the double-stranded DNA 601 passes through a pore 605 of a nanopore device 606, the nanopore device 606 can detect whether a fusion molecule is bound to the DNA 601 and whether the bound fusion molecule binds to an anti-HIV antibody 604.

FIG. 3B shows a fusion molecule that has an antibody analyte capture domain fused to a Azide reactive group through a PEG linker.

### Target Analytes and Ligands

In the present technology, a target analyte is detected or quantitated by virtue of its binding to a ligand in a fusion molecule that also binds to a polymer scaffold. A target analyte and a corresponding binding ligand with an analyte binding moiety can recognize and bind each other. For a larger analyte, there can be surface molecules or markers suitable for a ligand to bind (therefore the marker and the ligand form a binding pair).

Examples of binding pairs that enable binding between a target analyte and a ligand, but are not limited to, antigen/antibody (or antibody fragment); hormone, neurotransmitter, cytokine, growth factor or cell recognition molecule/receptor; and ion or element/chelate agent or ion binding protein, such as a calmodulin. The binding pairs can also be single-stranded nucleic acids having complementary sequences, enzymes and substrates, members of protein complex that bind each other, enzymes and cofactors, enzymes and one or more of their inhibitors (allosteric or otherwise), nucleic acid/protein, or cells or proteins detectable by cysteine-constrained peptides.

In some embodiments, the ligand is a protein, protein scaffold, peptide, aptamer (DNA or protein), nucleic acid (DNA or RNA), antibody fragment (Fab), chemically synthesized molecule, chemically reactive functional group or any other suitable structure that forms a binding pair with a target analyte.

Therefore, any target analyte in need of detection or quantitation, such as proteins, peptides, nucleic acids, chemical compounds, ions, and elements, can find a corresponding binding ligand. For the majority of proteins and nucleic acids, an antibody or a complementary sequence, or an aptamer can be readily prepared.

Likewise, binding ligands (such as antibodies and aptamers) can be readily found or prepared for analytes such as protein complexes and protein aggregates, protein/nucleic acid complexes, fragmented or fully assembled viruses, bacteria, cells, and cellular aggregates.

### Nanopore Devices

A nanopore device, as provided, includes at least a pore that forms an opening in a structure separating an interior space of the device into two volumes, and at least a sensor configured to identify objects (for example, by detecting changes in parameters indicative of objects) passing through the pore. Nanopore devices used for the methods described herein are also disclosed in PCT Publication WO/2013/012881, incorporated by reference in entirety.

The pore(s) in the nanopore device are of a nano scale or micro scale. In one aspect, each pore has a size that allows a small or large molecule or microorganism to pass. In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, the pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In some aspects, each pore is at least about 100 nm, 200 nm, 500 nm, 1000 nm, 2000 nm, 3000 nm, 5000 nm, 10000 nm, 20000 nm, or 30000 nm in diameter. In one aspect, the pore is no more than about 100000 nm in diameter. Alternatively, the pore is no more than about 50000 nm, 40000 nm, 30000 nm, 20000 nm, 10000 nm, 9000 nm, 8000 nm, 7000 nm, 6000 nm, 5000 nm, 4000 nm, 3000 nm, 2000 nm, or 1000 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the pore(s) in the nanopore device are of a larger scale for detecting large microorganisms or cells. In one aspect, each pore has a size that allows a large cell or microorganism to pass. In one aspect, each pore is at least about 100 nm in diameter. Alternatively, each pore is at least about 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, 1600 nm, 1700 nm, 1800 nm, 1900 nm, 2000 nm, 2500 nm, 3000 nm, 3500 nm, 4000 nm, 4500 nm, or 5000 nm in diameter.

In one aspect, the pore is no more than about 100,000 nm in diameter. Alternatively, the pore is no more than about 90,000 nm, 80,000 nm, 70,000 nm, 60,000 nm, 50,000 nm, 40,000 nm, 30,000 nm, 20,000 nm, 10,000 nm, 9000 nm, 8000 nm, 7000 nm, 6000 nm, 5000 nm, 4000 nm, 3000 nm, 2000 nm, or 1000 nm in diameter.

In one aspect, the pore has a diameter that is between about 100 nm and about 10000 nm, or alternatively between about 200 nm and about 9000 nm, or between about 300 nm and about 8000 nm, or between about 400 nm and about 7000 nm, or between about 500 nm and about 6000 nm, or between about 1000 nm and about 5000 nm, or between about 1500 nm and about 3000 nm.

In some aspects, the nanopore device further includes means to move a polymer scaffold across the pore and/or means to identify objects that pass through the pore. Further details are provided below, described in the context of a two-pore device.

Compared to a single-pore nanopore device, a two-pore device can be more easily configured to provide good control of speed and direction of the movement of the polymer scaffold across the pores.

In certain embodiments, the nanopore device includes a plurality of chambers, each chamber in communication with an adjacent chamber through at least one pore. Among these pores, two pores, namely a first pore and a second pore, are placed so as to allow at least a portion of a polymer scaffold to move out of the first pore and into the second pore. Further, the device includes a sensor capable of identifying the polymer scaffold during the movement. In one aspect, the identification entails identifying individual components of the polymer scaffold. In another aspect, the identification entails identifying fusion molecules and/or target analytes bound to the polymer scaffold. When a single sensor is employed, the single sensor may include two electrodes placed at both ends of a pore to measure an ionic current across the pore. In another embodiment, the single sensor comprises a component other than electrodes.

In one aspect, the device includes three chambers connected through two pores. Devices with more than three chambers can be readily designed to include one or more additional chambers on either side of a three-chamber device, or between any two of the three chambers. Likewise, more than two pores can be included in the device to connect the chambers.

In one aspect, there can be two or more pores between two adjacent chambers, to allow multiple polymer scaffolds to move from one chamber to the next simultaneously. Such a multi-pore design can enhance throughput of polymer scaffold analysis in the device.

In some aspects, the device further includes means to move a polymer scaffold from one chamber to another. In one aspect, the movement results in loading the polymer scaffold across both the first pore and the second pore at the same time. In another aspect, the means further enables the movement of the polymer scaffold, through both pores, in the same direction.

For instance, in a three-chamber two-pore device (a “two-pore” device), each of the chambers can contain an electrode for connecting to a power supply so that a separate voltage can be applied across each of the pores between the chambers.

In accordance with an embodiment of the present disclosure, provided is a device comprising an upper chamber, a middle chamber and a lower chamber, wherein the upper chamber is in communication with the middle chamber through a first pore, and the middle chamber is in communication with the lower chamber through a second pore. Such a device may have any of the dimensions or other characteristics previously disclosed in U.S. Publ. No. 2013-0233709, entitled Dual-Pore Device, which is herein incorporated by reference in its entirety.

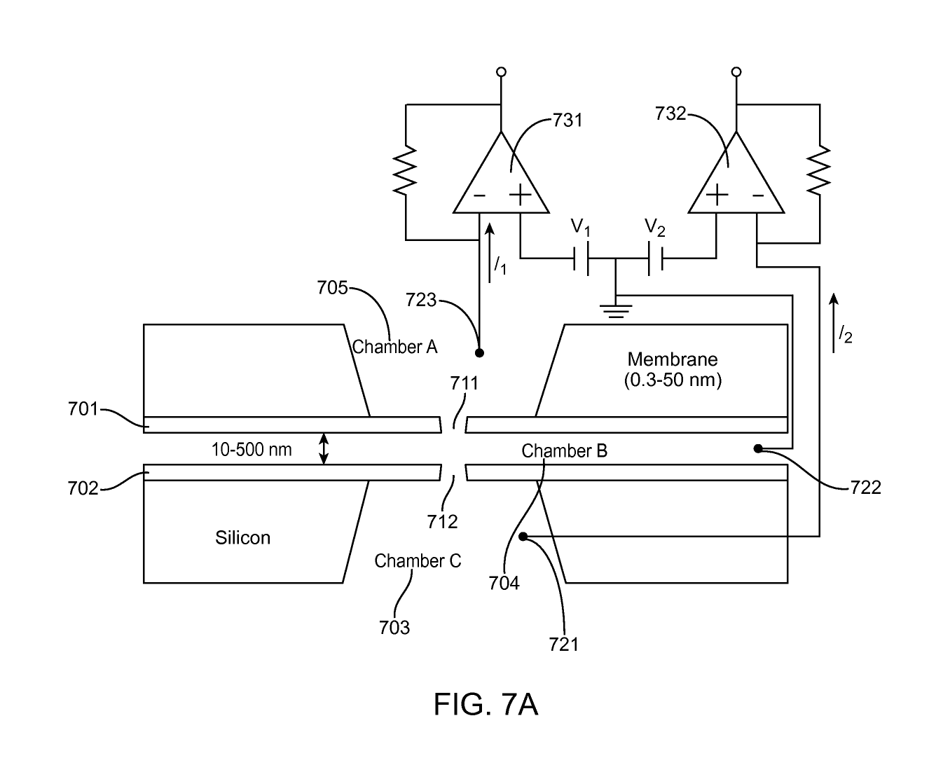

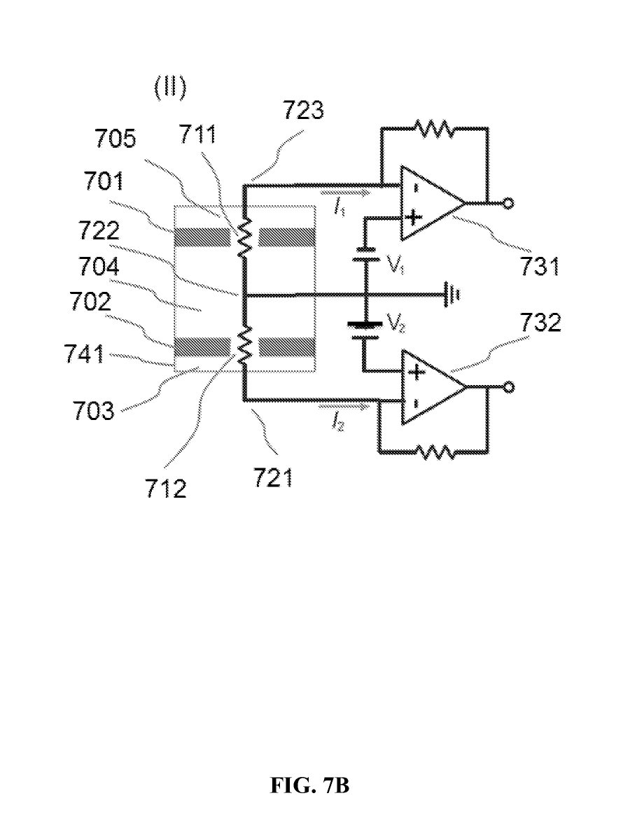

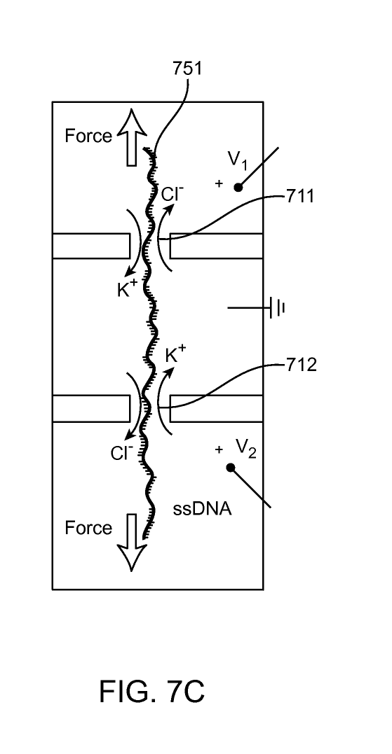

In some embodiments as shown in FIG. 7A, the device includes an upper chamber 705 (Chamber A), a middle chamber 704 (Chamber B), and a lower chamber 703 (Chamber C). The chambers are separated by two separating layers or membranes (701 and 702) each having a separate pore (711 or 712). Further, each chamber contains an electrode (721, 722 or 723) for connecting to a power supply. The annotation of upper, middle and lower chamber is in relative terms and does not indicate that, for instance, the upper chamber is placed above the middle or lower chamber relative to the ground, or vice versa.

Each of the pores 711 and 712 independently has a size that allows a small or large molecule or microorganism to pass. In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, the pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In other aspects, each pore is at least about 100 nm, 200 nm, 500 nm, 1000 nm, 2000 nm, 3000 nm, 5000 nm, 10000 nm, 20000 nm, or 30000 nm in diameter. In one aspect, each pore is 50,000 nm to 100,000 nm in diameter. In one aspect, the pore is no more than about 100000 nm in diameter. Alternatively, the pore is no more than about 50000 nm, 40000 nm, 30000 nm, 20000 nm, 10000 nm, 9000 nm, 8000 nm, 7000 nm, 6000 nm, 5000 nm, 4000 nm, 3000 nm, 2000 nm, or 1000 nm in diameter.

In some aspects, the pore has a substantially round shape. “Substantially round”, as used here, refers to a shape that is at least about 80 or 90% in the form of a cylinder. In some embodiments, the pore is square, rectangular, triangular, oval, or hexangular in shape.

Each of the pores 711 and 712 independently has a depth (i.e., a length of the pore extending between two adjacent volumes). In one aspect, each pore has a depth that is least about 0.3 nm. Alternatively, each pore has a depth that is at least about 0.6 nm, 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, or 90 nm.

In one aspect, each pore has a depth that is no more than about 100 nm. Alternatively, the depth is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm.

In one aspect, the pore has a depth that is between about 1 nm and about 100 nm, or alternatively, between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the nanopore extends through a membrane. For example, the pore may be a protein channel inserted in a lipid bilayer membrane or it may be engineered by drilling, etching, or otherwise forming the pore through a solid-state substrate such as silicon dioxide, silicon nitride, grapheme, or layers formed of combinations of these or other materials. In some aspects, the length or depth of the nanopore is sufficiently large so as to form a channel connecting two otherwise separate volumes. In some such aspects, the depth of each pore is greater than 100 nm, 200 nm, 300 nm, 400 nm, 500 nm, 600 nm, 700 nm, 800 nm, or 900 nm. In some aspects, the depth of each pore is no more than 2000 nm or 1000 nm.

In one aspect, the pores are spaced apart at a distance that is between about 10 nm and about 1000 nm. In some aspects, the distance between the pores is greater than 1000 nm, 2000 nm, 3000 nm, 4000 nm, 5000 nm, 6000 nm, 7000 nm, 8000 nm, or 9000 nm. In some aspects, the pores are spaced no more than 30000 nm, 20000 nm, or 10000 nm apart. In one aspect, the distance is at least about 10 nm, or alternatively, at least about 20 nm, 30 nm, 40 nm, 50 nm, 60 nm, 70 nm, 80 nm, 90 nm, 100 nm, 150 nm, 200 nm, 250 nm, or 300 nm. In another aspect, the distance is no more than about 1000 nm, 900 nm, 800 nm, 700 nm, 600 nm, 500 nm, 400 nm, 300 nm, 250 nm, 200 nm, 150 nm, or 100 nm.

In yet another aspect, the distance between the pores is between about 20 nm and about 800 nm, between about 30 nm and about 700 nm, between about 40 nm and about 500 nm, or between about 50 nm and about 300 nm

The two pores can be arranged in any position so long as they allow fluid communication between the chambers and have the prescribed size and distance between them. In one aspect, the pores are placed so that there is no direct blockage between them. Still, in one aspect, the pores are substantially coaxial, as illustrated in FIG. 7A.

In one aspect, as shown in FIG. 7A, the device, through the electrodes 721, 722, and 723 in the chambers 703, 704, and 705, respectively, is connected to one or more power supplies. In some aspects, the power supply includes a voltage-clamp or a patch-clamp, which can supply a voltage across each pore and measure the current through each pore independently. In this respect, the power supply and the electrode configuration can set the middle chamber to a common ground for both power supplies. In one aspect, the power supply or supplies are configured to apply a first voltage V₁ between the upper chamber 705 (Chamber A) and the middle chamber 704 (Chamber B), and a second voltage V₂ between the middle chamber 704 and the lower chamber 703 (Chamber C).

In some aspects, the first voltage V₁ and the second voltage V₂ are independently adjustable. In one aspect, the middle chamber is adjusted to be a ground relative to the two voltages. In one aspect, the middle chamber comprises a medium for providing conductance between each of the pores and the electrode in the middle chamber. In one aspect, the middle chamber includes a medium for providing a resistance between each of the pores and the electrode in the middle chamber. Keeping such a resistance sufficiently small relative to the nanopore resistances is useful for decoupling the two voltages and currents across the pores, which is helpful for the independent adjustment of the voltages.

Adjustment of the voltages can be used to control the movement of charged particles in the chambers. For instance, when both voltages are set in the same polarity, a properly charged particle can be moved from the upper chamber to the middle chamber and to the lower chamber, or the other way around, sequentially. In some aspects, when the two voltages are set to opposite polarity, a charged particle can be moved from either the upper or the lower chamber to the middle chamber and kept there.

The adjustment of the voltages in the device can be particularly useful for controlling the movement of a large molecule, such as a charged polymer scaffold, that is long enough to cross both pores at the same time. In such an aspect, the direction and the speed of the movement of the molecule can be controlled by the relative magnitude and polarity of the voltages as described below.

The device can contain materials suitable for holding liquid samples, in particular, biological samples, and/or materials suitable for nanofabrication. In one aspect, such materials include dielectric materials such as, but not limited to, silicon, silicon nitride, silicon dioxide, graphene, carbon nanotubes, TiO₂, HfO₂, Al₂O₃, or other metallic layers, or any combination of these materials. In some aspects, for example, a single sheet of graphene membrane of about 0.3 nm thick can be used as the pore-bearing membrane.

Devices that are microfluidic and that house two-pore microfluidic chip implementations can be made by a variety of means and methods. For a microfluidic chip comprised of two parallel membranes, both membranes can be simultaneously drilled by a single beam to form two concentric pores, though using different beams on each side of the membranes is also possible in concert with any suitable alignment technique. In general terms, the housing ensures sealed separation of Chambers A-C. In one aspect as shown in FIG. 7B, the housing would provide minimal access resistance between the voltage electrodes 721, 722, and 723 and the nanopores 711 and 712, to ensure that each voltage is applied principally across each pore.

In one aspect, the device includes a microfluidic chip (labeled as “Dual-core chip”) is comprised of two parallel membranes connected by spacers. Each membrane contains a pore drilled by a single beam through the center of the membrane. Further, the device preferably has a Teflon® housing for the chip. The housing ensures sealed separation of Chambers A-C and provides minimal access resistance for the electrode to ensure that each voltage is applied principally across each pore.

More specifically, the pore-bearing membranes can be made with transmission electron microscopy (TEM) grids with a 5-100 nm thick silicon, silicon nitride, or silicon dioxide windows. Spacers can be used to separate the membranes, using an insulator, such as SU-8, photoresist, PECVD oxide, ALD oxide, ALD alumina, or an evaporated metal material, such as Ag, Au, or Pt, and occupying a small volume within the otherwise aqueous portion of Chamber B between the membranes. A holder is seated in an aqueous bath that is comprised of the largest volumetric fraction of Chamber B. Chambers A and C are accessible by larger diameter channels (for low access resistance) that lead to the membrane seals.

A focused electron or ion beam can be used to drill pores through the membranes, naturally aligning them. The pores can also be sculpted (shrunk) to smaller sizes by applying a correct beam focusing to each layer. Any single nanopore drilling method can also be used to drill the pair of pores in the two membranes, with consideration to the drill depth possible for a given method and the thickness of the membranes. Predrilling a micro-pore to a prescribed depth and then a nanopore through the remainder of the membranes is also possible to further refine the membrane thickness.

In another aspect, the insertion of biological nanopores into solid-state nanopores to form a hybrid pore can be used in either or both pores in the two-pore method. The biological pore can increase the sensitivity of the ionic current measurements, and is useful when only single-stranded polynucleotides are to be captured and controlled in the two-pore device, e.g., for sequencing.

By virtue of the voltages present at the pores of the device, charged molecules can be moved through the pores between chambers. Speed and direction of the movement can be controlled by the magnitude and polarity of the voltages. Further, because each of the two voltages can be independently adjusted, the direction and speed of the movement of a charged molecule can be finely controlled in each chamber.

One example concerns a charged polymer scaffold, such as a DNA, having a length that is longer than the combined distance that includes the depth of both pores plus the distance between the two pores. For example, a 1000 by dsDNA is about 340 nm in length, and would be substantially longer than the 40 nm spanned by two 10 nm-deep pores separated by 20 nm. In a first step, the polynucleotide is loaded into either the upper or the lower chamber. By virtue of its negative charge under a physiological condition at a pH of about 7.4, the polynucleotide can be moved across a pore on which a voltage is applied. Therefore, in a second step, two voltages, in the same polarity and at the same or similar magnitudes, are applied to the pores to move the polynucleotide across both pores sequentially.

At about the time when the polynucleotide reaches the second pore, one or both of the voltages can be changed. Since the distance between the two pores is selected to be shorter than the length of the polynucleotide, when the polynucleotide reaches the second pore, it is also in the first pore. A prompt change of polarity of the voltage at the first pore, therefore, will generate a force that pulls the polynucleotide away from the second pore as illustrated in FIG. 7C.

Assuming that the two pores have identical voltage-force influence and |V₁|=|V₂|+δV, the value δV>0 (or <0) can be adjusted for tunable motion in the V₁| (or V₂) direction. In practice, although the voltage-induced force at each pore will not be identical with V₁=V₂, calibration experiments can identify the appropriate bias voltage that will result in equal pulling forces for a given two-pore chip; and variations around that bias voltage can then be used for directional control.

If, at this point, the magnitude of the voltage-induced force at the first pore is less than that of the voltage-induced force at the second pore, then the polynucleotide will continue crossing both pores towards the second pore, but at a lower speed. In this respect, it is readily appreciated that the speed and direction of the movement of the polynucleotide can be controlled by the polarities and magnitudes of both voltages. As will be further described below, such a fine control of movement has broad applications.

Accordingly, in one aspect, provided is a method for controlling the movement of a charged polymer scaffold through a nanopore device. The method entails (a) loading a sample comprising a charged polymer scaffold in one of the upper chamber, middle chamber or lower chamber of the device of any of the above embodiments, wherein the device is connected to one or more power supplies for providing a first voltage between the upper chamber and the middle chamber, and a second voltage between the middle chamber and the lower chamber; (b) setting an initial first voltage and an initial second voltage so that the polymer scaffold moves between the chambers, thereby locating the polymer scaffold across both the first and second pores; and (c) adjusting the first voltage and the second voltage so that both voltages generate force to pull the charged polymer scaffold away from the middle chamber (voltage-competition mode), wherein the two voltages are different in magnitude, under controlled conditions, so that the charged polymer scaffold moves across both pores in either direction and in a controlled manner.

To establish the voltage-competition mode in step (c), the relative force exerted by each voltage at each pore is to be determined for each two-pore device used, and this can be done with calibration experiments by observing the influence of different voltage values on the motion of the polynucleotide, which can be measured by sensing known-location and detectable features in the polynucleotide, with examples of such features detailed later in this disclosure. If the forces are equivalent at each common voltage, for example, then using the same voltage value at each pore (with common polarity in upper and lower chambers relative to grounded middle chamber) creates a zero net motion in the absence of thermal agitation (the presence and influence of Brownian motion is discussed below). If the forces are not equivalent at each common voltage, achieving equal forces involves the identification and use of a larger voltage at the pore that experiences a weaker force at the common voltage. Calibration for voltage-competition mode can be done for each two-pore device, and for specific charged polymers or molecules whose features influence the force when passing through each pore.

In one aspect, the sample containing the charged polymer scaffold is loaded into the upper chamber and the initial first voltage is set to pull the charged polymer scaffold from the upper chamber to the middle chamber and the initial second voltage is set to pull the polymer scaffold from the middle chamber to the lower chamber. Likewise, the sample can be initially loaded into the lower chamber, and the charged polymer scaffold can be pulled to the middle and the upper chambers.

In another aspect, the sample containing the charged polymer scaffold is loaded into the middle chamber; the initial first voltage is set to pull the charged polymer scaffold from the middle chamber to the upper chamber; and the initial second voltage is set to pull the charged polymer scaffold from the middle chamber to the lower chamber.

In one aspect, the adjusted first voltage and second voltage at step (c) are about 10 times to about 10,000 times as high, in magnitude, as the difference/differential between the two voltages. For instance, the two voltages can be 90 mV and 100 mV, respectively. The magnitude of the two voltages, about 100 mV, is about 10 times of the difference/differential between them, 10 mV. In some aspects, the magnitude of the voltages is at least about 15 times, 20 times, 25 times, 30 times, 35 times, 40 times, 50 times, 100 times, 150 times, 200 times, 250 times, 300 times, 400 times, 500 times, 1000 times, 2000 times, 3000 times, 4000 times, 5000 times, 6000 times, 7000 times, 8000 times or 9000 times as high as the difference/differential between them. In some aspects, the magnitude of the voltages is no more than about 10000 times, 9000 times, 8000 times, 7000 times, 6000 times, 5000 times, 4000 times, 3000 times, 2000 times, 1000 times, 500 times, 400 times, 300 times, 200 times, or 100 times as high as the difference/differential between them.

In one aspect, real-time or on-line adjustments to the first voltage and the second voltage at step (c) are performed by active control or feedback control using dedicated hardware and software, at clock rates up to hundreds of megahertz. Automated control of the first or second or both voltages is based on feedback of the first or second or both ionic current measurements.

### Sensors

As discussed above, in various aspects, the nanopore device further includes one or more sensors to carry out the identification of the binding status of the binding motifs.

The sensors used in the device can be any sensor suitable for identifying a target analyte, such as a polymer. For instance, a sensor can be configured to identify the polymer (e.g., a polymer scaffold) by measuring a current, a voltage, a pH value, an optical feature, or residence time associated with the polymer. In other aspects, the sensor may be configured to identify one or more individual components of the polymer or one or more components bound to the polymer. The sensor may be formed of any component configured to detect a change in a measurable parameter where the change is indicative of the polymer, a component of the polymer, or preferably, a component bound to the polymer. In one aspect, the sensor includes a pair of electrodes placed at two sides of a pore to measure an ionic current across the pore when a molecule or other entity, in particular a polymer scaffold, moves through the pore. In certain aspects, the ionic current across the pore changes measurably when a polymer scaffold segment passing through the pore is bound to a probe, such as a label, a fusion molecule and/or fusion molecule-target analyte complex. Such changes in current may vary in predictable, measurable ways corresponding with, for example, the presence, absence, and/or size of the fusion molecules and target analytes present.

In a preferred embodiment, the sensor comprises electrodes which apply voltage and are used to measure current across the nanopore. Translocations of molecules through the nanopore provides electrical impedance (Z) which affects current through the nanopore according to Ohm's Law, V=IZ, where V is voltage applied, I is current through the nanopore, and Z is impedance. The result when a molecule translocates through a nanopore in an electrical field (e.g., under an applied voltage) is an electrical signal that may be correlated to the molecule passing through the nanopore upon further analysis of the current signal.

When residence time measurements from the electrical signal are used, the size of the component can be correlated to the specific component based on the length of time it takes to pass through the sensing device.

In an embodiment, a sensor is provided in the nanopore device that measures an optical feature of the polymer, a component (or unit) of the polymer, or a component bound to the polymer. One example of such measurement includes the identification of an absorption band unique to a particular unit by infrared (or ultraviolet) spectroscopy.

In some embodiments, the sensor is an electric sensor. In some embodiments, the sensor detects a fluorescent detection means when the target analyte or the detectable label passing through has a unique fluorescent signature. A radiation source at the outlet of the pore can be used to detect that signature.

Analysis of Data from Nanopore Detection

Described herein are methods of encoding one or more bit(s) of information by placing one or more molecules along a polymer scaffold so that information encoded in the polymer scaffold can be retrieved by passing the polymer scaffold through a nanopore and examining the current impedance signatures curves.

A molecule that is used on a polymer for the sole purpose of storing information is called a “label.” A label is considered “unique” if it causes a signature curve that can be differentiated against other labels (synthetic) or molecules (natural) on that same polymer. A single polymer scaffold can contain one or more labels to represent increasingly more complex information. Therefore, a synthetic polymers bound to one or more labels reside in a reservoir without the presence of natural molecules, this method can be used to store arbitrary amounts of static information for later recall.

A method of data retrieval of data encoded in a polymer scaffold is performed in a device that contains one or more nanopores, and a chamber with synthetic polymers that contain labels. A voltage is applied, causing negatively charged molecules, including the polymer scaffold, to pass through the nanopore. As molecules pass through the pore, events are generated, and the data is analyzed by the software to discern the presence of known signature curves. If a (portion of the) signature curve matches one of the known labels, the rest of the event is analyzed for more signature curves, and they are assembled in the same order in which they assembled on the polymer. The software determines if/how to translate the information captured into whatever intended purpose the software serves. (e.g., different signature curves may map to different letters of an alphabet, or pixel values, or MIDI data.)

When synthetic polymers are used in reservoirs that also contain molecules found in nature, the synthetic polymer must be designed in such a manner that the event is assured to be different from that which would be generated by any of the natural molecules in the same reservoir. A synthetic polymer may also have additional sites that have binding molecules intended to capture natural analytes that may reside in the reservoir.

In an embodiment, the method of identifying an analyte from a bulk solution is performed on a device that contains one or more nanopores, and a chamber with synthetic polymers that contain labels and fusion molecules intended to capture one or more analytes. A microfluidic channel may be included in the device that allows sample fluid from a natural source to enter into the reservoir chamber. As the molecules from the sample interact with the synthetic polymers, target analytes will bind with the fusion molecules. A voltage is then applied to the sample mixture, causing negatively charged molecules, such as polymer scaffolds, to pass through the nanopore. As molecules pass through the pore, events are generated, and the data is analyzed by the software to discern the presence of known signature curves.

If the software does not identify any of the signature curves from the set of known labels, the entire event is discarded. If a signature curve matches one of the known labels, the rest of the event is analyzed for more signature curves. If the software determines that the polymer has a binding molecule, that molecule's signature curve is analyzed to see if a target analyte was attached to the binder.

In another embodiment optical signals may be used instead of current impedance measurements to discern the presence of molecules along the polymer scaffold. In an embodiment, the method of detecting optical signals from a polymer scaffold to read data encoded on the polymer scaffold is performed in a nanopore device. Voltage is applied to drive negatively charged polymers through the nanopore. An optical sensor is used in the device to capture an optical measurement within a fixed field of view that may reside at or adjacent to the nanopore. The optical measurement comprises a measure of light detected within a fixed period of time. This measurement may include, but not be limited to, one or more of individual values, such as color, luminescence, and intensity. The method can be used to detect a tagged molecule in a chemical complex that has been modified in such a manner to generate an optical signal that an optical sensor will detect, providing a particular optical measurement.

An “optical event” is a set of optical measurements captured by the sensor from a single polymer scaffold that may contain one or more tagged molecules. Because the sensor cannot discern between the beginning and end of a polymer using optics, the ends of the polymer may be detected by current impedance measurements to determine when a polymer enters (e.g., when the measurement's y value deviates beyond an open channel threshold, or adding tagged molecules that will produce a known optical measurement when bound at each end of the polymer. An optical signature is a collection of optical measurements within an optical event where the software analyzes them in such a manner that it determines it has read a unique abstract value. Since a polymer may have one or more molecules attached to it, an event may contain one or more signatures. A symbol is the assembly of one or more optical signatures within an event so as to comprise a single abstraction. E.g., “red, green, red, green” may equate to the letter “A”

In several of the embodiments, the electrical signal provided may be compared against a database that correlates a molecule or complex with an electrical signal. This molecule or complex may be any of the entities discussed herein as capable of detecting via current impedance upon translocation through the nanopore, or other methods of detection, such as optical measurements. A database may be generated by reading the electrical signals provided by a homogenous population. Analysis of a homogenous population of polymer scaffolds bound to probes, which may further be bound to analytes or other entities is useful for assessing the variation in signal pattern generated and determining a reference signal for that coded molecule. Events and electrical signals from a sample combined with the same polymer scaffold and probes can then be analyzed and compared to the database comprising the reference signals correlated to an analyte or polymer scaffold identification and/or quantitation.

### EXAMPLES

The present technology is further defined by reference to the following example and experiments. It will be apparent to those skilled in the art that many modifications may be practiced without departing from the scope of the current invention

### Example 1

### DNA Alone in Solid-State Nanopore Experiment

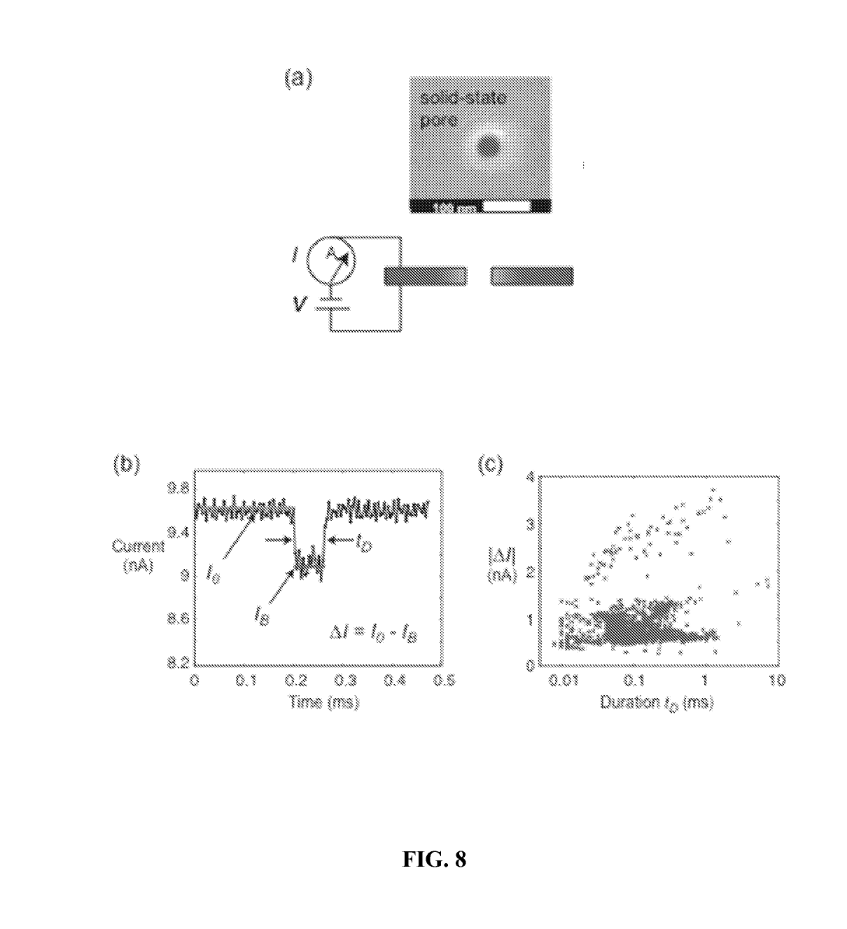

Nanopore instruments use a sensitive voltage-clamp amplifier to apply a voltage V across the pore while measuring the ionic current l₀ through the open pore (FIG. 8, panel (a)). When a single charged molecule such as a double-stranded DNA (dsDNA) is captured and driven through the pore by electrophoresis (FIG. 8, panel (b)), the measured current shifts from l₀ to l_{B}, and the shift amount Δ/=l₀−l_{B} and duration t_{D} are used to characterize the event. After recording many events during an experiment, distributions of the events (FIG. 8, panel (c)) are analyzed to characterize the corresponding molecule. In this way, nanopores provide a simple, label-free, purely electrical single-molecule method for biomolecular sensing.

In the DNA experiment shown in FIG. 8, the single nanopore fabricated in silicon nitride (SiN) substrate is a 40 nm diameter pore in 100 nm thick SiN membrane (FIG. 8, panel (a)). In FIG. 8(b), the representative current trace shows a blockade event caused by a 5.6 kb dsDNA passing in a single file manner (unfolded) through an 11 nm diameter nanopore in 10 nm thick SiN at 200 mV and 1M KCI. The mean open channel current is l₀=9.6 nA, with mean event amplitude l_{B}=9.1 nA, and duration t_{D}=0.064 ms. The amplitude shift is Δ/=l₀−l_{B}=0.5 nA. In FIG. 6C, the scatter plot shows |Δ/| vs. t_{D} for all 1301 events recorded over 16 minutes.

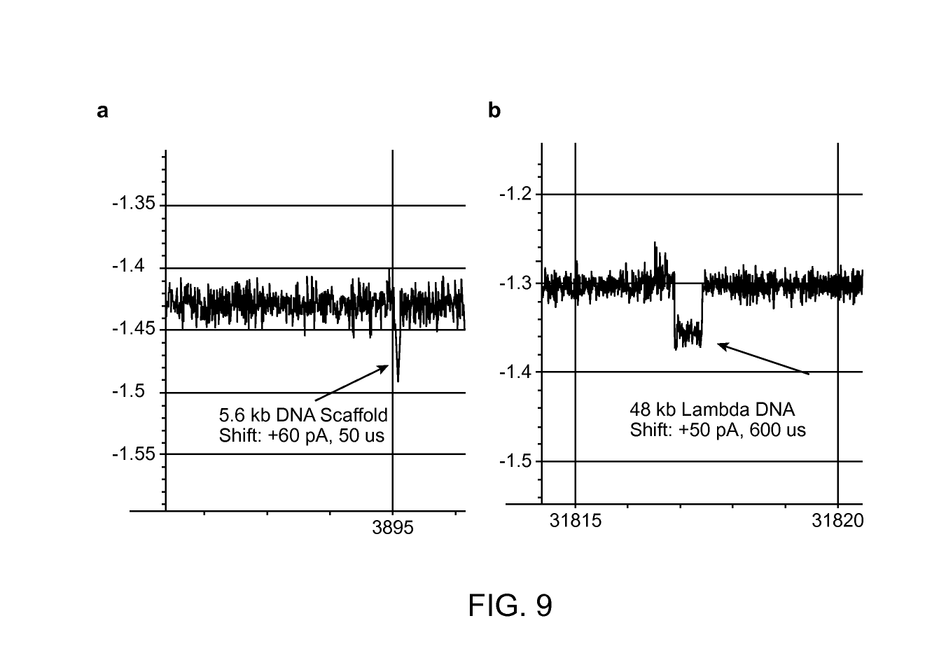

In the DNA experiment shown in FIG. 9, dsDNA alone causes current enhancement events at 100 mM KCI. This was shown in the published research of Smeets, Ralph M M, et al. “Salt dependence of ion transport and DNA translocation through solid-state nanopores.” Nano Letters 6.1 (2006): 89-95). The study showed that, while the amplitude shift Δ/=l₀−l_{B}>0 for KCI concentration above 0.4 M, the shift has opposite polarity (Δ/<0) for KCI concentration below 0.4 M. As this is a negative voltage experiment (−200 mV) with KCI concentration below 0.4 M, we see that the DNA event has the same polarity (416) relative to the baseline (415) as shown in FIG. 4C.

### Example 2

### Binding of PNA to dsDNA Scaffold and Detection in a Nanopore

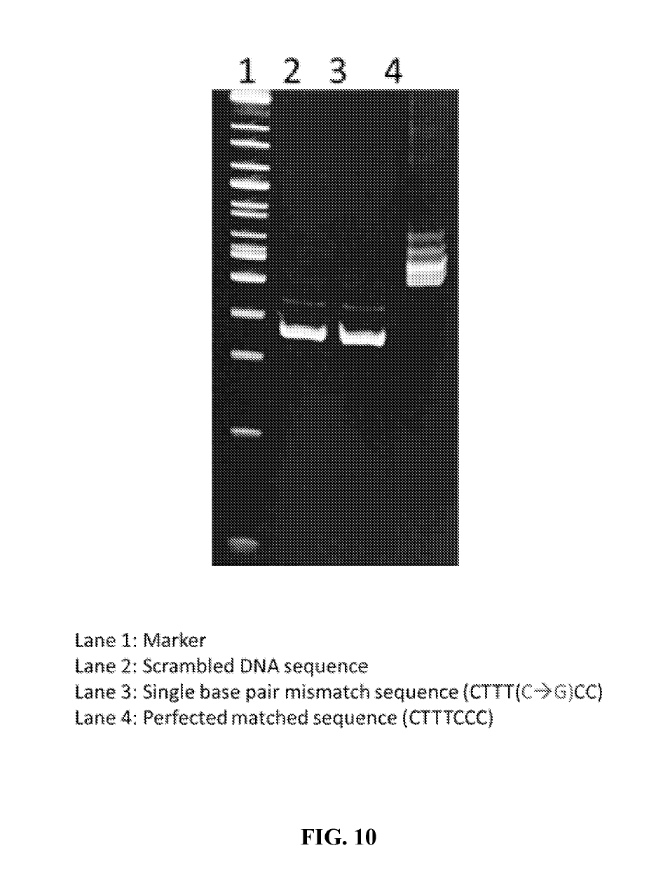

To show that the bisPNA molecule is specific for its target sequence, binding experiments were performed using a scrambled 324 by dsDNA fragment, a 324 bp dsDNA fragment with a complementary sequence to the bisPNA except for a single base pair mismatch sequence, and a 324 bp dsDNA fragment with a perfectly matched complementary sequence. FIG. 10 shows that only the perfect match sequence shows bisPNA binding. Thus, the bisPNA scaffold binding domain binds to the label binding domain on the bisPNA with high stringency and selectivity.

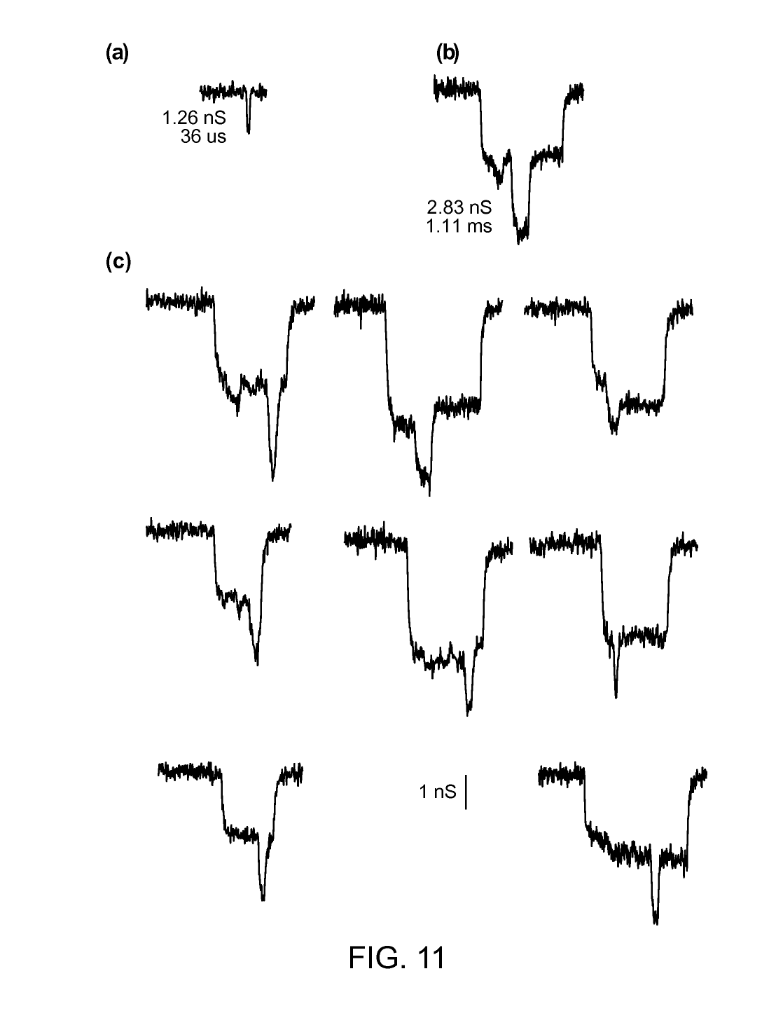

In addition, a 4-6 nm nanopore in a nanopore device is capable of detecting the bisPNA label on the dsDNA scaffold. As shown in FIG. 11, a nanopore assay as described herein is capable of detecting the (a) absence, or (b,c) presence of a bis-PNA label to the target sequence of a 324 bp dsDNA. With the 7 bp target sequence located in the middle, representative events show a distinct pattern not observed otherwise.

### Example 3

### Detection of PNA Bound to a Detectable Tag in a Nanopore

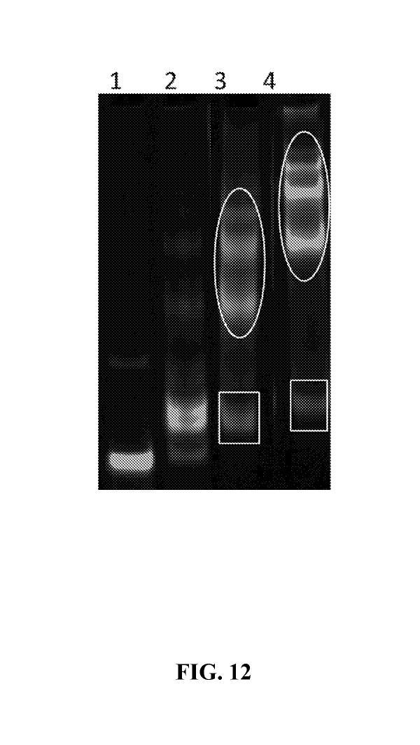

The formation of label-DNA complexes, where the label comprises a detectable tag was shown as follows. dsDNA was incubated with bis-PNA molecules comprising either 5 kDa and 10 kDa PEG as a detectable tag. Formation of the label-dsDNA complex was observed in a gel as shown in FIG. 12. In lane 1, DNA alone is run as a control. A 324 bp DNA fragment was bound by bisPNA that contained no PEG (lane 2), bound by a bisPNA that contained 5 kDa PEG (lane 3), or bound by a bisPNA that had a 10 kDa PEG conjugated. The triple banding pattern in lanes 3 and 4 (circled) are due to the different conformations the PNA takes when binding. The lowest band in lanes 3 and 4 (square) is likely DNA bound by PNA that was not PEG labeled.

Next, we determined whether we could detect the PNA labels with PEG detectable tags bound to dsDNA polymer scaffold in a nanopore assay. We ran a sample comprising 324 bp dsDNA with a bis-PNA binding domain and either (a) ZERO-payload, (b) 5 kDa PEG-payload, or (c) 10 kDa PEG-payload bits attached to bis-PNA.

As shown in FIG. 13 a nanopore was able to discriminate DNA alone and DNA bound by bisPNA with ZERO, PEG 5 k and PEG 10 k payloads bound to 324 bp dsDNA. With the 7 bp target sequence located in the middle, representative events show a distinct pattern observed distinct for each bit, using 15-35 nm diameter nanopores. Each PNA has 3 PEGs of the stated size as the detectable tag. All events are on a common vertical scale for current amplitude. These events were collected from the same experiment, showing simultaneous bit discrimination

### Example 4

### Probe Multiplexing

We determined that an individual polymer scaffold is capable of reliably binding multiple labels. Such labels can generate distinct electrical signatures when bound to a scaffold and passed through an appropriately designed nanopore. We ran DNA, DNA bound to a single PNA attached to PEG 5 k, and DNA bound to a single PNA attached to PEG 5 k.

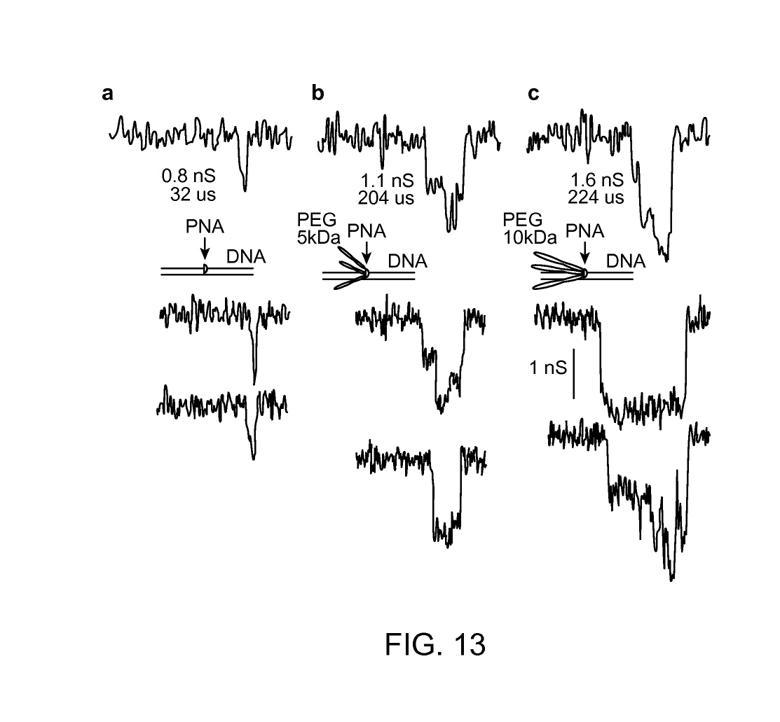

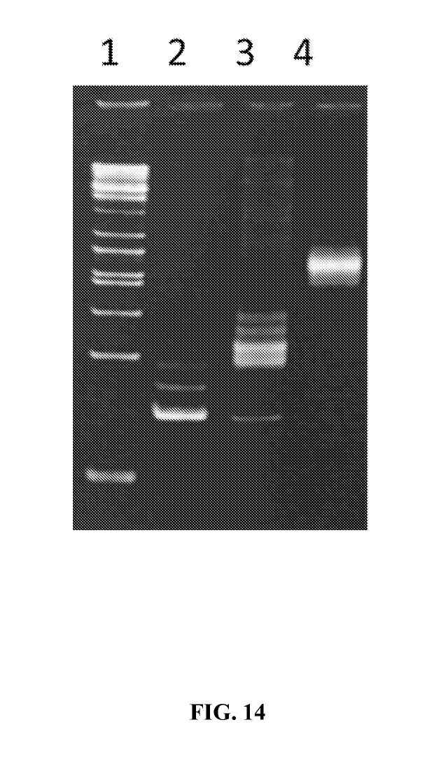

Gel shift shows gammaPNA-PEG 5 kDa can bind to the same sequence. As shown in FIG. 14, a gel shift assay shows that a single or two gammaPNA-PEG 5 kDa can bind to the same fragment molecule. Lane 1: Marker. Lane 2: DNA fragment only. Lane 3: DNA fragment+1×PNA-5 k. Lane 4: DNA fragment+2×PNA-5 k. Thus, multiple probes, such as labels or fusion molecules can bind to the same scaffold to allow multiplexing.

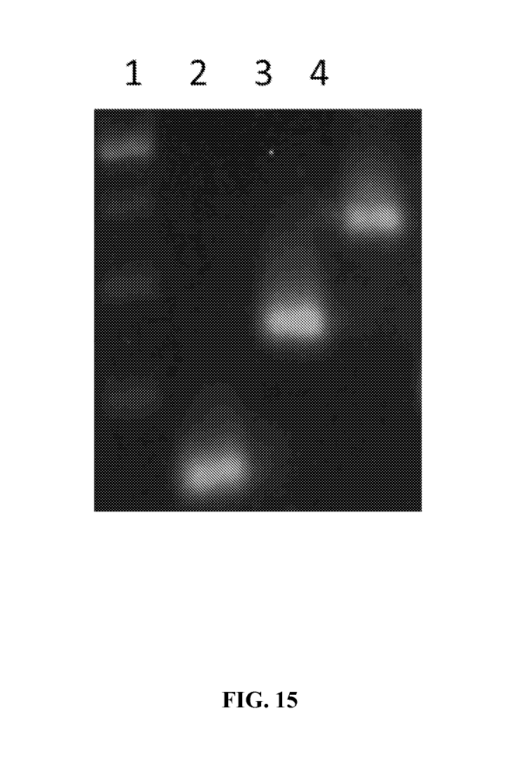

We also showed that a polymer scaffold is capable of binding a plurality of monostreptavidin proteins as probes. We generated DNA fragments comprising biotinylated ends using biotinylated PCR, then incubated the DNA with monostreptavidin. The gel shift shown in FIG. 15 shows a DNA fragment can be reliably tagged with a plurality of monostreptavidin proteins. Lane 1 shows a marker. Lane 2 shows DNA fragment only. Lane 3 shows DNA fragment+1× monostreptavidin. Lane 4 shows DNA fragment+2× monostreptavidin. Thus, DNA fragments generated by biotinylated PCR primers can be bound by a plurality of streptavidin protein labels.

Detection of Multiple Sites of a Sequence in 5.6 kb dsDNA

A linear 5.6 kbp dsDNA molecule was engineered to contain a unique 12 bp sequence (uSeq1) interspersed at 25 sites within the DNA. The purpose of this repetition is to boost the sensing signal for each scaffold, since the more occupied PNA sites there are, the longer the nanopore current is impeded, yielding a more easily detected signature.

Instead of using bis-PNA as the sequence-specific binding molecule, we used the smaller and more versatile γPNA. Positive detection and localization of these smaller PNAs is possible, but required a precision sub-4 nm pore, and was shown to work with a salt gradient. In the absence of a salt gradient and with a larger pore (11 nm diameter, 10 nm membrane), we sought to demonstrate positive detection of the presence of the label by adding a detectable tag to each PNA. To provide the option of increasing label size, the PNA had three biotin molecules incorporated via coupling to free amines on the backbone Lysine amino acid.

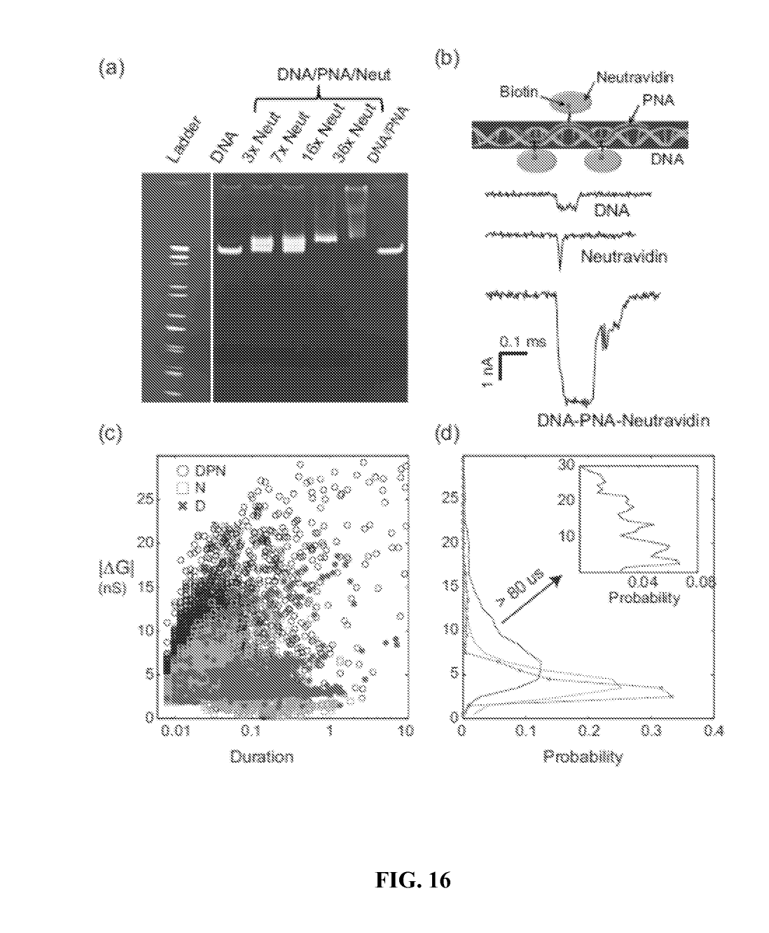

This DNA-PNA-Neutravidin (DPN) reagent was tested in using a nanopore 11 nm in diameter formed by dielectric breakdown in a 10 nm membrane. FIG. 16, panels (b-d) show data comparing ΔG versus duration distributions for events from three separate experiments conducted sequentially on the same pore: DNA alone, Neutravidin alone, and DPN reagents.

The largest ΔG events in the DPN experiment are attributed to DPN complexes (FIG. 16, panel (b)), providing a simple criteria for tagging events as having the target 12 by sequence. The mathematical criteria derived above can be used to assess confidence in detection. Using the criteria ΔG>20 nS, 390 of the events in the DPN experiment are tagged resulting in

Q(p)=9.29%. In the prior control experiments, only 0.46% of D and 0.16% of N events are detected. Applying the mathematical criteria above, with Q=Fraction of N-flagged events, the 99% confidence interval is Q=9.29+/−1.15% for this data set. Since 9.29%>0.46% (the max false-positive %) well within the 99% confidence interval for Q, we have a positive test result, and in under 8 minutes of data gathering. In fact the same 99% confidence is achieved for this data set with only the first 60 seconds of the data. The gel shift (FIG. 16, panel (a)) shows that scaffold DNA migration is retarded in a Neutravidin dependent manner and guided us to using the 10× concentration in this preliminary experiment, as it appeared all DNA is bound and a nearly homogenous population is created.

### Example 5

### Detection of an Analyte in Human Blood Using a dsDNA Scaffold

We ran 1:20 diluted samples of human blood (whole and serum) with a control polymer scaffold in the nanopore device. conducted nanopore experiments that incorporates human blood (whole and serum) with a control molecule. Several generic events from the current impedance data from background molecules in the mixed sample were generated. Rec-A coated DNA was then added to the sample to test the ability to distinguish RecA coated DNA from background molecules based on the electrical signal generated upon translocation through the nanopore. The ability to detect RecA coated DNA in the sample using the nanopore was hindered due to overlap between electrical signals from RecA coated DNA and background molecules in the blood sample.

However, a labeled polymeric scaffold, such as PNA with a detectable tag attached to dsDNA, provides an electrical signal that is unique from the background molecules in blood when these molecules translocate through the nanopore under an applied voltage. Therefore, we will generate a polymer scaffold that comprises label binding domains and fusion molecule binding domains. The label binding domains attach to labels optionally comprising a detectable tag that will provide a unique electrical signal to distinguish the polymer scaffold from background molecules. Then, the detection of a target analyte by an electrical signal present on the current event generated by the polymer scaffold can be performed by analysis of the electrical signal provided by the attached or unattached fusion molecule.

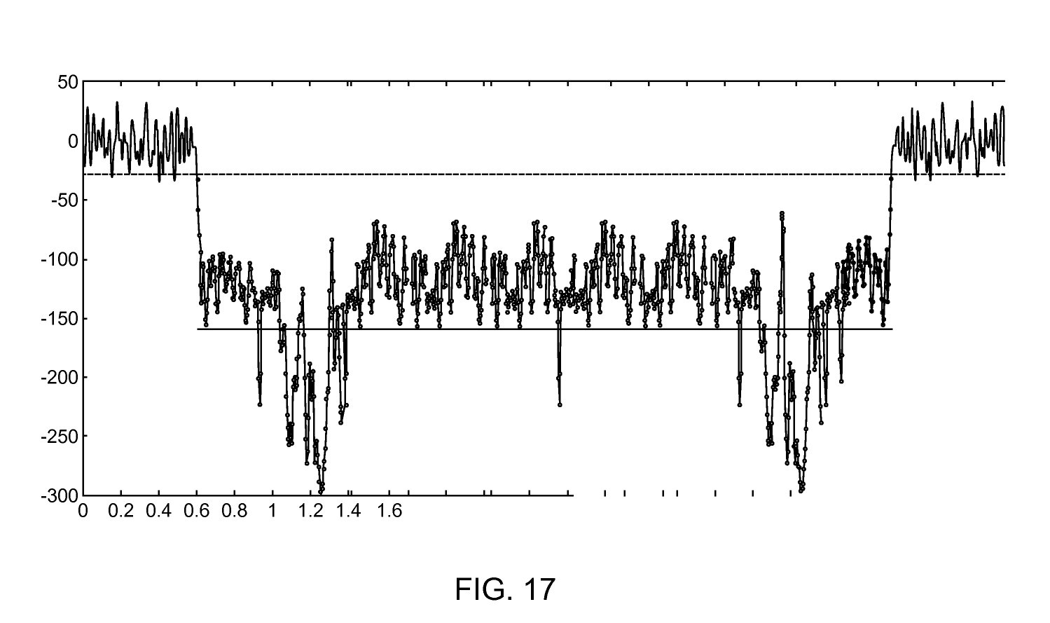

Therefore, the use of labels and binding label domains on the polymer scaffold will be used to identify scaffolds that have one or more target analytes. FIG. 17 shows a prototype illustration of an electrical signal generated upon the translocation of a polymer scaffold with PNA molecules attached to 5K PEGs on either end of the polymer scaffold, with a fusion molecules and target analyte in the middle, through the nanopore. The unique signature provided by this construct is not present in bulk samples. The molecule is too long and unique features on either end are too uniform, so that there is very low probability that an overlapping electrical signal would be produced by a natural molecule that is not the engineered polymer scaffold. Therefore, the fusion molecule in the center of the electrical signal in FIG. 17 may be specifically analyzed for the absence or presence of an analyte.

To optimize the performance of the system so that it can be computationally simple, we can use known algorithms to smooth out specific events or electrical signals, such as those correlated with a polymer scaffold translocation. For example, we can use moving averages, Bollinger Bands, among others, to infer the shape of data. We will use these algorithms to compare shapes of all events from the nanopore and quickly isolate those that resolve within a threshold. We can also use a unique compression algorithm tuned for certain electrical signals to reduce storage size of the data.

Additional polymer scaffolds for analyte detection comprising a defined sequence of label binding domains and at least one fusion molecule binding domain for analyte detection will be generated using this method, which will allow us to discriminate from all background events. Additionally, little or no sample prep is needed for assays where the target analytes in solution in the sample. However, some sample prep to extract that targets embedded in, e.g., cells or soil may be performed to liquefy the sample or isolate certain portions of the sample.

It is to be understood that while the invention has been described in conjunction with the above embodiments, the foregoing description and examples are intended to illustrate and not limit the scope of the invention. Other aspects, advantages and modifications within the scope of the invention will be apparent to those skilled in the art to which the invention pertains.

### Example 6

### Detection of Label and Presence or Absence of Target Using a dsDNA Scaffold

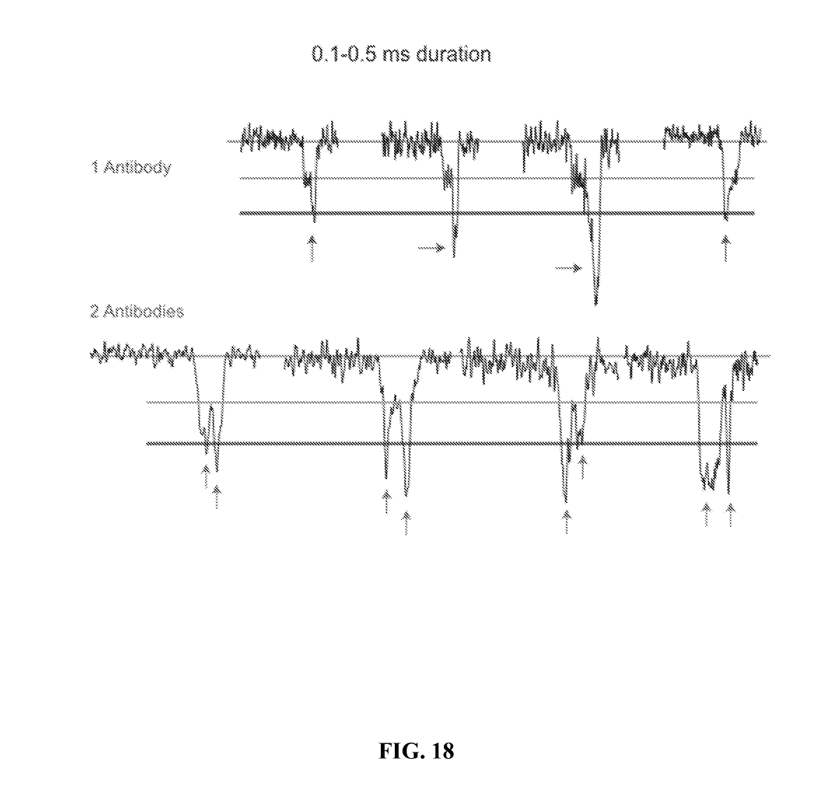

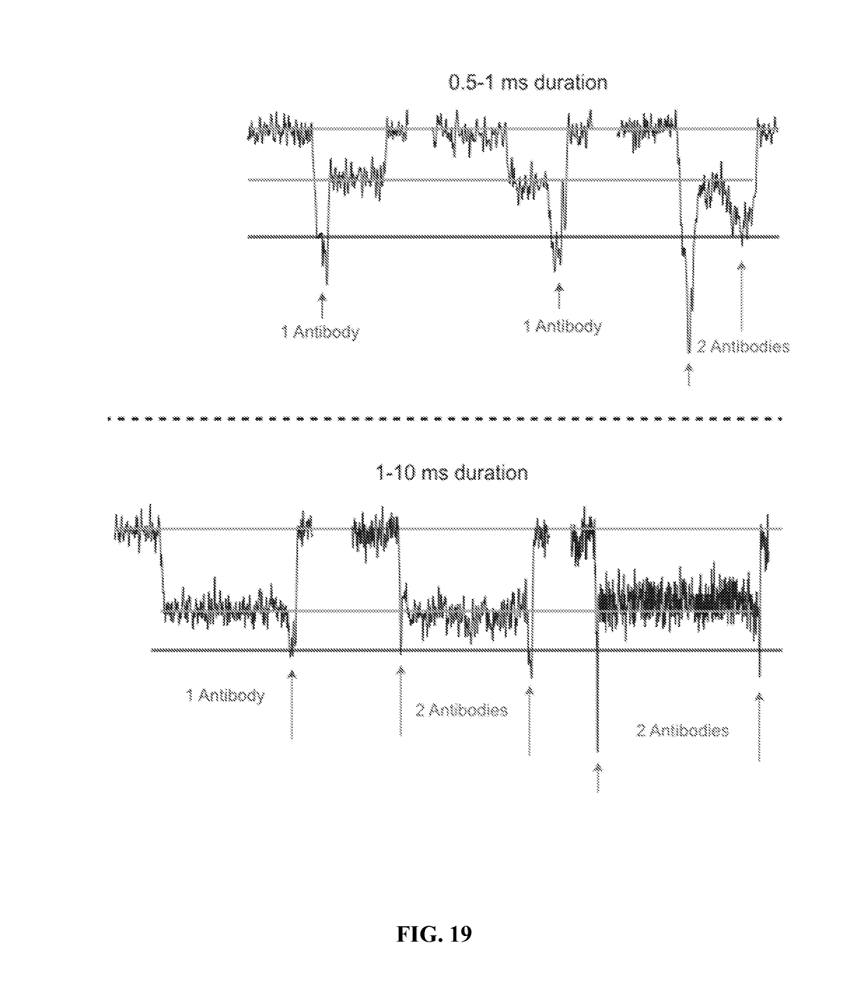

The polymer scaffold comprises probe binding domains that may reside on the ends of the DNA as chemical modification to which labels or analyte detection molecules are chemically tethered or bound. FIG. 18 shows a dsDNA scaffold with events 0.1-0.5 ms, and with a single antibody acting as a label at one end, and the absence or presence of a separate target analyte antibody at the other end. Event signatures have a single “spike” when only the label antibody is present, and two “spikes” when the target analyte antibody is present, signaling detection of the target for that molecule. These spikes are identified by automated algorithms, that quantitate the spikes as have distinct amplitude levels and durations. The algorithm can work on shorter or longer duration events, and with steps of shorter or longer duration within events. FIG. 19 shows a dsDNA scaffold with longer lasting events (0.5-10 ms), still with a single antibody acting as a label at one end, and the absence or presence of a separate target analyte antibody at the other end. As before, event signatures have a single “spike” when only the label antibody is present, and two “spikes” when the target analyte antibody is present, signaling detection of the target for that molecule. The detections and event classifications are all done by algorithms in software, and can be in real-time or offline after experimentation.

1. A compound comprising a polymeric scaffold, a fusion molecule attached to said polymeric scaffold, and a label attached to said polymeric scaffold.

2. The compound of claim 1, wherein said attachment is covalent.

3. The compound of claim 1, wherein said attachment is non-covalent.

4. The compound of claim 1, wherein said polymeric scaffold comprises dsDNA.

5. The compound of claim 1, wherein said polymeric scaffold is dsDNA.

6. The compound of claim 1, wherein said polymeric scaffold comprises a fusion molecule binding domain.

7. The compound of claim 6, wherein said fusion molecule binding domain is bound to a fusion molecule.

8. The compound of claim 1, wherein said polymeric scaffold comprises a label binding domain.

9. The compound of claim 8, wherein said label binding domain is attached to a label.

10. The compound of claim 1, wherein said fusion molecule comprises an antibody, an antibody fragment, an epitope, a hormone, a neurotransmitter, a cytokine, a growth factor, a cell recognition molecule, a nucleic acid, a peptide, an aptamer (nucleic acid, protein, PNA, or combination thereof), or a receptor.

11. The compound of claim 1, wherein said fusion molecule comprises PNA bound to a molecule comprising a target binding moiety.

12. The compound of claim 11, wherein said molecule comprising a target binding moiety is an antibody or an aptamer.

13. The compound of claim 1, wherein said fusion molecule comprises RecA or VspR.

14. The compound of claim 1, wherein said fusion molecule comprises protein, BNA, LNA, CRISPR, TALEN, or DNA.

15. The compound of claim 1, wherein said polymeric scaffold comprises at least two unique fusion molecules attached to said polymeric scaffold.

16. The compound of claim 15, wherein said at least two unique fusion molecules are each bound to a unique target analyte.

17. The compound of claim 1, wherein said fusion molecule is bound to a target analyte.

18. The compound of claim 17, wherein said target analyte comprises a protein, a peptide, a polynucleotide, a chemical compound, an ion, or an element.

19. The compound of claim 18, wherein said target analyte comprises a protein complex or aggregate, a protein/nucleic acid complex, a fragmented or fully assembled virus, a bacterium, a cell, or a cellular aggregate.

20. The compound of claim 1, wherein said fusion molecule is bound to a target analyte via one or more intermediary molecules.

21. The compound of claim 1, wherein said fusion molecule comprises a target binding domain capable of binding to the target analyte, and wherein said fusion molecule comprises a scaffold binding domain capable of binding to the polymer scaffold at a specific target.

22. The compound of claim 21, wherein said specific target comprises a specific polymer sequence.

23. The compound of claim 1, wherein said label comprises PNA.

24. The compound of claim 1, wherein said label comprises a detectable tag.

25. The compound of claim 24, wherein said detectable tag comprises PEG.

26. The compound of claim 1, wherein said label comprises an oligonucleotide, a PNA, a polypeptide, a protein, or an aptamer.

27. The compound of claim 1, wherein said label comprises a scaffold binding domain capable of binding to the polymer scaffold at a specific target.

28. The compound of claim 27, wherein said specific target comprises a specific polymer sequence.

29. The compound of claim 1, wherein said polymeric scaffold comprises a DNA molecule, a PNA molecule, an RNA molecule, or a polypeptide molecule.

30. The compound of claim 1, wherein said polymer scaffold is attached to a plurality of labels, wherein at least two labels have a unique size, shape, hydrophobicity or charge that renders each capable of generating detectably distinct electrical signals in a nanopore device.

31. A method of detecting a target analyte suspected to be present in a mixed sample, comprising:
a. loading a polymer scaffold, a fusion molecule, a label, and a mixed sample suspected to contain a target analyte into a device comprising a nanopore that separates an interior space of the device into two volumes, under conditions that allow said label to bind to said polymer scaffold, that allow said fusion molecule to bind to said polymer scaffold, and that allow said fusion molecule to bind to said target analyte,
i. wherein said polymer scaffold is attached to at least one fusion molecule,
ii. wherein said polymer scaffold is attached to at least one label, and
iii. wherein said fusion molecule comprises a target binding domain capable of binding to the target analyte;
b. configuring the device to pass the polymer scaffold in any orientation through the nanopore from one volume to the other volume; and
c. collecting an electrical signal correlated to passage of said polymeric scaffold in any orientation through the nanopore.

32. The method of claim 31, wherein said attachments are covalent.

33. The method of claim 31, wherein said attachments are non-covalent.

34. The method of claim 31, wherein said polymer scaffold comprises at least one fusion molecule binding domain capable of binding to the fusion molecule.

35. The method of claim 31, wherein said polymer scaffold comprises at least one label binding domain capable of binding to the label.

36. The method of claim 31, wherein said fusion molecule comprises a scaffold binding domain capable of binding to the polymer scaffold at a first target.

37. The method of claim 31, wherein said label comprises a scaffold binding domain capable of binding to the polymer scaffold at a second target

38. The method of claim 31, wherein said polymer scaffold comprises dsDNA.

39. The method of claim 31, wherein said polymeric scaffold is dsDNA.

40. The method of claim 31, wherein said fusion molecule provides a unique and detectable electrical signal in a target analyte-bound state as compared to a target analyte-unbound state upon translocation through the nanopore when bound to said polymer scaffold.

41. The method of claim 31, wherein said fusion molecule comprises PNA bound to a molecule comprising a target binding moiety.

42. The method of claim 41, wherein said molecule comprising a target binding moiety comprises an antibody.

43. The method of claim 31, wherein said fusion molecule comprises RecA or VspR.

44. The method of claim 31, wherein said mixed sample comprises an environmental sample or a biological sample.

45. The method of claim 31, wherein said mixed sample comprises whole blood, red blood cells, white blood cells, hair, nails, swabs, urine, sputum, saliva, semen, lymphatic fluid, amniotic fluid, cerebrospinal fluid, peritoneal effusions, pleural effusions, fluid from cysts, synovial fluid, vitreous humor, aqueous humor, bursa fluid, eye washes, eye aspirates, plasma, serum, pulmonary lavage, lung aspirates, liver, spleen, kidney, lung, intestine, brain, heart, muscle, pancreas, primary cell lines, secondary cell lines, or any combination thereof.

46. The method of claim 31, wherein said mixed sample comprises food, water, soil, or waste.

47. The method of claim 31, wherein said device comprises at least two nanopores in series, and wherein said polymer scaffold is simultaneously in said at least two nanopores during translocation.

48. A method of analyzing data to detect the presence or absence of a target analyte in a mixed sample, comprising
a. obtaining an electrical signal from an event generated by a nanopore analysis of a mixture, wherein said mixture comprises a sample suspected of containing a target analyte and background molecules capable of generating background electrical signals, a polymer scaffold bound to a label and a fusion molecule capable of binding to said target analyte;
b. analyzing said electrical signal to detect the presence or absence of a first signature indicating detection of a label attached to the polymer scaffold;
c. analyzing said electrical signal to detect the presence of a second signature indicating detection of a fusion molecule that is bound to said target analyte, or a third signature indicating detection of a fusion molecule that is not bound to said target analyte, wherein the background electrical signals are distinct from the first, second, and third signatures, wherein the presence of said first and said second signatures indicates the presence of said target analyte in said mixed sample, and wherein the presence of said first and said third signatures indicate the absence of said target analyte in said mixed sample.

49. The method of claim 48, wherein said polymer scaffold comprises dsDNA.

50. The method of claim 48, wherein said polymer scaffold is dsDNA.

51. The method of claim 48, wherein said polymer scaffold comprises at least one fusion molecule binding domain capable of binding to the fusion molecule.

52. The method of claim 48, wherein said polymer scaffold comprises at least one label binding domain capable of binding to the label.

53. The method of claim 48, wherein said fusion molecule comprises a scaffold binding domain capable of binding to the polymer scaffold at a first target.

54. The method of claim 48, wherein said label comprises a scaffold binding domain capable of binding to the polymer scaffold at a second target

55. The method of claim 48, wherein analyzing said event to detect the presence or absence of a first signature comprises comparing said electrical signal to a database comprising a correlation of a signature to a label attached to the polymer scaffold.

56. The method of claim 48, wherein analyzing said event to detect the presence of a second signature comprises comparing said electrical signal to a database comprising a correlation of a signature to a fusion molecule attached to the polymer scaffold that is bound to the target analyte.

57. The method of claim 48, wherein analyzing said event to detect the presence of a third signature comprises comparing said electrical signal to a database comprising a correlation of a signature to a fusion molecule attached to the polymer scaffold that is not bound to the target analyte.

58. The method of claim 48, wherein said mixed sample comprises an environmental sample or a biological sample.

59. The method of claim 48, wherein said mixed sample comprises whole blood, red blood cells, white blood cells, hair, nails, swabs, urine, sputum, saliva, semen, lymphatic fluid, amniotic fluid, cerebrospinal fluid, peritoneal effusions, pleural effusions, fluid from cysts, synovial fluid, vitreous humor, aqueous humor, bursa fluid, eye washes, eye aspirates, plasma, serum, pulmonary lavage, lung aspirates, liver, spleen, kidney, lung, intestine, brain, heart, muscle, pancreas, primary cell lines, secondary cell lines, or any combination thereof.

60. The method of claim 48, wherein said mixed sample comprises food, water, soil, or waste.

61. A kit, comprising (a) a polymer scaffold, (b) a label capable of binding to said polymer scaffold, (c) and a fusion molecule capable of binding to a target ligand and to said polymer scaffold.

62. A method for identifying binding sequences on a polymer scaffold, comprising:
a. providing a polymer scaffold comprising a label binding domain;
b. loading said polymer scaffold and a label configured to bind to said label binding domain into a device comprising a nanopore that separates an interior space of the device into two volumes, under conditions that allow said label to bind to said label binding sequence;
c. configuring the device to pass the polymer scaffold through the nanopore from one volume to the other volume; and
d. detecting an electrical signal correlated to passage of said polymeric scaffold through the nanopore.

63. The method of claim 62, wherein said polymer scaffold comprises dsDNA.

64. The method of claim 62, wherein said polymer scaffold is dsDNA.

65. The method of claim 62, wherein said electrical signal comprises a measure of current impedance in said nanopore over time.

66. The method of claim 62, wherein said polymer comprises a plurality of label binding sequences, wherein said label binding sequences each bind to a unique label, and wherein each label bound to said nanopore provides a unique electrical signal upon translocation through said nanopore.

67. The method of claim 62, wherein said polymer comprises a plurality of label binding sequences, wherein said label binding sequences each bind to a unique label, and wherein each label provides a unique electrical signature.

68. The method of claim 62, wherein said device comprises at least two nanopores in series, and wherein said polymer scaffold is simultaneously spanning said at least two nanopores during translocation.

69. A method of analyzing data encoded in a polymer scaffold, comprising:
a. obtaining an electrical signal from an event generated by a nanopore analysis of a mixture, wherein said mixture comprises a polymer scaffold comprising at least one label binding domain, and a label configured to bind to said label binding domain;
b. analyzing said electrical signal to detect the presence of a first signature indicating the presence of a label binding sequence on said polymer scaffold.

70. The method of claim 69, wherein said polymer scaffold comprises dsDNA.

71. The method of claim 69, wherein said polymer scaffold is dsDNA.

72. A kit comprising (a) two or more labels each having different size, charge and/or shape and comprising a polymeric scaffold and (b) a nanopore device comprising a nanopore that separates and connects two volumes in the nanopore device, wherein the nanopore device is configured to identify each of the labels when the label is bound to said polymeric scaffold and said polymeric scaffold translocates through said nanopore.
